SEQUENCE LISTING (911 sequences)

SEQ ID NO: 1 - DNA, 10 bp, Artificial Sequence
  cDNA sequence NM 003140.2
gttgaggggg

SEQ ID NO: 2 - DNA, 10 bp, Artificial Sequence
  cDNA sequence NM 003140.2
ttgagggggt

SEQ ID NO: 3 - DNA, 10 bp, Artificial Sequence
  cDNA sequence NM 003140.2
tgagggggtg

SEQ ID NO: 4 - DNA, 10 bp, Artificial Sequence
  cDNA sequence NM 003140.2
gagggggtgt

SEQ ID NO: 5 - DNA, 10 bp, Artificial Sequence
  cDNA sequence NM 003140.2
agggggtgtt

SEQ ID NO: 6 - DNA, 10 bp, Artificial Sequence
  cDNA sequence NM 003140.2
gggggtgttg

SEQ ID NO: 7 - DNA, 10 bp, Artificial Sequence
  cDNA sequence NM 003140.2
ggggtgttga

SEQ ID NO: 8 - DNA, 10 bp, Artificial Sequence
  cDNA sequence NM 003140.2
gggtgttgag

SEQ ID NO: 9 - DNA, 10 bp, Artificial Sequence
  cDNA sequence NM 003140.2
ggtgttgagg

SEQ ID NO: 10 - DNA, 10 bp, Artificial Sequence
  cDNA sequence NM 003140.2
gtgttgaggg

SEQ ID NO: 11 - DNA, 10 bp, Artificial Sequence
  cDNA sequence NM 003140.2
tgttgagggc

SEQ ID NO: 12 - DNA, 10 bp, Artificial Sequence
  cDNA sequence NM 003140.2
gttgagggcg

SEQ ID NO: 13 - DNA, 10 bp, Artificial Sequence
  cDNA sequence NM 003140.2
ttgagggcgg

SEQ ID NO: 14 - DNA, 10 bp, Artificial Sequence
  cDNA sequence NM 003140.2
tgagggcgga

SEQ ID NO: 15 - DNA, 10 bp, Artificial Sequence
  cDNA sequence NM 003140.2
gagggcggag

SEQ ID NO: 16 - DNA, 10 bp, Artificial Sequence
  cDNA sequence NM 003140.2
agggcggaga

SEQ ID NO: 17 - DNA, 10 bp, Artificial Sequence
  cDNA sequence NM 003140.2
gggcggagaa

SEQ ID NO: 18 - DNA, 10 bp, Artificial Sequence
  cDNA sequence NM 003140.2
ggcggagaaa

SEQ ID NO: 19 - DNA, 10 bp, Artificial Sequence
  cDNA sequence NM 003140.2
gcggagaaat

SEQ ID NO: 20 - DNA, 10 bp, Artificial Sequence
  cDNA sequence NM 003140.2
cggagaaatg

SEQ ID NO: 21 - DNA, 10 bp, Artificial Sequence
  cDNA sequence NM 003140.2
ggagaaatgc

SEQ ID NO: 22 - DNA, 10 bp, Artificial Sequence
  cDNA sequence NM 003140.2
gagaaatgca

SEQ ID NO: 23 - DNA, 10 bp, Artificial Sequence
  cDNA sequence NM 003140.2
agaaatgcaa

SEQ ID NO: 24 - DNA, 10 bp, Artificial Sequence
  cDNA sequence NM 003140.2
gaaatgcaag

SEQ ID NO: 25 - DNA, 10 bp, Artificial Sequence
  cDNA sequence NM 003140.2
aaatgcaagt

SEQ ID NO: 26 - DNA, 10 bp, Artificial Sequence
  cDNA sequence NM 003140.2
aatgcaagtt

SEQ ID NO: 27 - DNA, 10 bp, Artificial Sequence
  cDNA sequence NM 003140.2
atgcaagttt

SEQ ID NO: 28 - DNA, 10 bp, Artificial Sequence
  cDNA sequence NM 003140.2
tgcaagtttc

SEQ ID NO: 29 - DNA, 10 bp, Artificial Sequence
  cDNA sequence NM 003140.2
gcaagtttca

SEQ ID NO: 30 - DNA, 10 bp, Artificial Sequence
  cDNA sequence NM 003140.2
caagtttcat

SEQ ID NO: 31 - DNA, 10 bp, Artificial Sequence
  cDNA sequence NM 003140.2
aagtttcatt

SEQ ID NO: 32 - DNA, 10 bp, Artificial Sequence
  cDNA sequence NM 003140.2
agtttcatta

SEQ ID NO: 33 - DNA, 10 bp, Artificial Sequence
  cDNA sequence NM 003140.2
gtttcattac

SEQ ID NO: 34 - DNA, 10 bp, Artificial Sequence
  cDNA sequence NM 003140.2
tttcattaca

SEQ ID NO: 35 - DNA, 10 bp, Artificial Sequence
  cDNA sequence NM 003140.2
ttcattacaa

SEQ ID NO: 36 - DNA, 10 bp, Artificial Sequence
  cDNA sequence NM 003140.2
tcattacaaa

SEQ ID NO: 37 - DNA, 10 bp, Artificial Sequence
  cDNA sequence NM 003140.2
cattacaaaa

SEQ ID NO: 38 - DNA, 10 bp, Artificial Sequence
  cDNA sequence NM 003140.2
attacaaaag

SEQ ID NO: 39 - DNA, 10 bp, Artificial Sequence
  cDNA sequence NM 003140.2
ttacaaaagt

SEQ ID NO: 40 - DNA, 10 bp, Artificial Sequence
  cDNA sequence NM 003140.2
tacaaaagtt

SEQ ID NO: 41 - DNA, 10 bp, Artificial Sequence
  cDNA sequence NM 003140.2
acaaaagtta

SEQ ID NO: 42 - DNA, 10 bp, Artificial Sequence
  cDNA sequence NM 003140.2
caaaagttaa

SEQ ID NO: 43 - DNA, 10 bp, Artificial Sequence
  cDNA sequence NM 003140.2
aaaagttaac

SEQ ID NO: 44 - DNA, 10 bp, Artificial Sequence
  cDNA sequence NM 003140.2
aaagttaacg

SEQ ID NO: 45 - DNA, 10 bp, Artificial Sequence
  cDNA sequence NM 003140.2
aagttaacgt

SEQ ID NO: 46 - DNA, 10 bp, Artificial Sequence
  cDNA sequence NM 003140.2
agttaacgta

SEQ ID NO: 47 - DNA, 10 bp, Artificial Sequence
  cDNA sequence NM 003140.2
gttaacgtaa

SEQ ID NO: 48 - DNA, 10 bp, Artificial Sequence
  cDNA sequence NM 003140.2
ttaacgtaac

SEQ ID NO: 49 - DNA, 10 bp, Artificial Sequence
  cDNA sequence NM 003140.2
taacgtaaca

SEQ ID NO: 50 - DNA, 10 bp, Artificial Sequence
  cDNA sequence NM 003140.2
aacgtaacaa

SEQ ID NO: 51 - DNA, 10 bp, Artificial Sequence
  cDNA sequence NM 003140.2
acgtaacaaa

SEQ ID NO: 52 - DNA, 10 bp, Artificial Sequence
  cDNA sequence NM 003140.2
cgtaacaaag

SEQ ID NO: 53 - DNA, 10 bp, Artificial Sequence
  cDNA sequence NM 003140.2
gtaacaaaga

SEQ ID NO: 54 - DNA, 10 bp, Artificial Sequence
  cDNA sequence NM 003140.2
taacaaagaa

SEQ ID NO: 55 - DNA, 10 bp, Artificial Sequence
  cDNA sequence NM 003140.2
aacaaagaat

SEQ ID NO: 56 - DNA, 10 bp, Artificial Sequence
  cDNA sequence NM 003140.2
acaaagaatc

SEQ ID NO: 57 - DNA, 10 bp, Artificial Sequence
  cDNA sequence NM 003140.2
caaagaatct

SEQ ID NO: 58 - DNA, 10 bp, Artificial Sequence
  cDNA sequence NM 003140.2
aaagaatctg

SEQ ID NO: 59 - DNA, 10 bp, Artificial Sequence
  cDNA sequence NM 003140.2
aagaatctgg

SEQ ID NO: 60 - DNA, 10 bp, Artificial Sequence
  cDNA sequence NM 003140.2
agaatctggt

SEQ ID NO: 61 - DNA, 10 bp, Artificial Sequence
  cDNA sequence NM 003140.2
gaatctggta

SEQ ID NO: 62 - DNA, 10 bp, Artificial Sequence
  cDNA sequence NM 003140.2
aatctggtag

SEQ ID NO: 63 - DNA, 10 bp, Artificial Sequence
  cDNA sequence NM 003140.2
atctggtaga

SEQ ID NO: 64 - DNA, 10 bp, Artificial Sequence
  cDNA sequence NM 003140.2
tctggtagaa

SEQ ID NO: 65 - DNA, 10 bp, Artificial Sequence
  cDNA sequence NM 003140.2
ctggtagaag

SEQ ID NO: 66 - DNA, 10 bp, Artificial Sequence
  cDNA sequence NM 003140.2
tggtagaagt

SEQ ID NO: 67 - DNA, 10 bp, Artificial Sequence
  cDNA sequence NM 003140.2
ggtagaagtg

SEQ ID NO: 68 - DNA, 10 bp, Artificial Sequence
  cDNA sequence NM 003140.2
gtagaagtga

SEQ ID NO: 69 - DNA, 10 bp, Artificial Sequence
  cDNA sequence NM 003140.2
tagaagtgag

SEQ ID NO: 70 - DNA, 10 bp, Artificial Sequence
  cDNA sequence NM 003140.2
agaagtgagt

SEQ ID NO: 71 - DNA, 10 bp, Artificial Sequence
  cDNA sequence NM 003140.2
gaagtgagtt

SEQ ID NO: 72 - DNA, 10 bp, Artificial Sequence
  cDNA sequence NM 003140.2
aagtgagttt

SEQ ID NO: 73 - DNA, 10 bp, Artificial Sequence
  cDNA sequence NM 003140.2
agtgagtttt

SEQ ID NO: 74 - DNA, 10 bp, Artificial Sequence
  cDNA sequence NM 003140.2
gtgagttttg

SEQ ID NO: 75 - DNA, 10 bp, Artificial Sequence
  cDNA sequence NM 003140.2
tgagttttgg

SEQ ID NO: 76 - DNA, 10 bp, Artificial Sequence
  cDNA sequence NM 003140.2
gagttttgga

SEQ ID NO: 77 - DNA, 10 bp, Artificial Sequence
  cDNA sequence NM 003140.2
agttttggat

SEQ ID NO: 78 - DNA, 10 bp, Artificial Sequence
  cDNA sequence NM 003140.2
gttttggata

SEQ ID NO: 79 - DNA, 10 bp, Artificial Sequence
  cDNA sequence NM 003140.2
ttttggatag

SEQ ID NO: 80 - DNA, 10 bp, Artificial Sequence
  cDNA sequence NM 003140.2
tttggatagt

SEQ ID NO: 81 - DNA, 10 bp, Artificial Sequence
  cDNA sequence NM 003140.2
ttggatagta

SEQ ID NO: 82 - DNA, 10 bp, Artificial Sequence
  cDNA sequence NM 003140.2
tggatagtaa

SEQ ID NO: 83 - DNA, 10 bp, Artificial Sequence
  cDNA sequence NM 003140.2
ggatagtaaa

SEQ ID NO: 84 - DNA, 10 bp, Artificial Sequence
  cDNA sequence NM 003140.2
gatagtaaaa

SEQ ID NO: 85 - DNA, 10 bp, Artificial Sequence
  cDNA sequence NM 003140.2
atagtaaaat

SEQ ID NO: 86 - DNA, 10 bp, Artificial Sequence
  cDNA sequence NM 003140.2
tagtaaaata

SEQ ID NO: 87 - DNA, 10 bp, Artificial Sequence
  cDNA sequence NM 003140.2
agtaaaataa

SEQ ID NO: 88 - DNA, 10 bp, Artificial Sequence
  cDNA sequence NM 003140.2
gtaaaataag

SEQ ID NO: 89 - DNA, 10 bp, Artificial Sequence
  cDNA sequence NM 003140.2
taaaataagt

SEQ ID NO: 90 - DNA, 10 bp, Artificial Sequence
  cDNA sequence NM 003140.2
aaaataagtt

SEQ ID NO: 91 - DNA, 10 bp, Artificial Sequence
  cDNA sequence NM 003140.2
aaataagttt

SEQ ID NO: 92 - DNA, 10 bp, Artificial Sequence
  cDNA sequence NM 003140.2
aataagtttc

SEQ ID NO: 93 - DNA, 10 bp, Artificial Sequence
  cDNA sequence NM 003140.2
ataagtttcg

SEQ ID NO: 94 - DNA, 10 bp, Artificial Sequence
  cDNA sequence NM 003140.2
taagtttcga

SEQ ID NO: 95 - DNA, 10 bp, Artificial Sequence
  cDNA sequence NM 003140.2
aagtttcgaa

SEQ ID NO: 96 - DNA, 10 bp, Artificial Sequence
  cDNA sequence NM 003140.2
agtttcgaac

SEQ ID NO: 97 - DNA, 10 bp, Artificial Sequence
  cDNA sequence NM 003140.2
gtttcgaact

SEQ ID NO: 98 - DNA, 10 bp, Artificial Sequence
  cDNA sequence NM 003140.2
tttcgaactc

SEQ ID NO: 99 - DNA, 10 bp, Artificial Sequence
  cDNA sequence NM 003140.2
ttcgaactct

SEQ ID NO: 100 - DNA, 10 bp, Artificial Sequence
  cDNA sequence NM 003140.2
tcgaactctg

SEQ ID NO: 101 - DNA, 10 bp, Artificial Sequence
  cDNA sequence NM 003140.2
cgaactctgg

SEQ ID NO: 102 - DNA, 10 bp, Artificial Sequence
  cDNA sequence NM 003140.2
gaactctggc

SEQ ID NO: 103 - DNA, 10 bp, Artificial Sequence
  cDNA sequence NM 003140.2
aactctggca

SEQ ID NO: 104 - DNA, 10 bp, Artificial Sequence
  cDNA sequence NM 003140.2
actctggcac

SEQ ID NO: 105 - DNA, 10 bp, Artificial Sequence
  cDNA sequence NM 003140.2
ctctggcacc

SEQ ID NO: 106 - DNA, 10 bp, Artificial Sequence
  cDNA sequence NM 003140.2
tctggcacct

SEQ ID NO: 107 - DNA, 10 bp, Artificial Sequence
  cDNA sequence NM 003140.2
ctggcacctt

SEQ ID NO: 108 - DNA, 10 bp, Artificial Sequence
  cDNA sequence NM 003140.2
tggcaccttt

SEQ ID NO: 109 - DNA, 10 bp, Artificial Sequence
  cDNA sequence NM 003140.2
ggcacctttc

SEQ ID NO: 110 - DNA, 10 bp, Artificial Sequence
  cDNA sequence NM 003140.2
gcacctttca

SEQ ID NO: 111 - DNA, 10 bp, Artificial Sequence
  cDNA sequence NM 003140.2
cacctttcaa

SEQ ID NO: 112 - DNA, 10 bp, Artificial Sequence
  cDNA sequence NM 003140.2
acctttcaat

SEQ ID NO: 113 - DNA, 10 bp, Artificial Sequence
  cDNA sequence NM 003140.2
cctttcaatt

SEQ ID NO: 114 - DNA, 10 bp, Artificial Sequence
  cDNA sequence NM 003140.2
ctttcaattt

SEQ ID NO: 115 - DNA, 10 bp, Artificial Sequence
  cDNA sequence NM 003140.2
tttcaatttt

SEQ ID NO: 116 - DNA, 10 bp, Artificial Sequence
  cDNA sequence NM 003140.2
ttcaattttg

SEQ ID NO: 117 - DNA, 10 bp, Artificial Sequence
  cDNA sequence NM 003140.2
tcaattttgt

SEQ ID NO: 118 - DNA, 10 bp, Artificial Sequence
  cDNA sequence NM 003140.2
caattttgtc

SEQ ID NO: 119 - DNA, 10 bp, Artificial Sequence
  cDNA sequence NM 003140.2
aattttgtcg

SEQ ID NO: 120 - DNA, 10 bp, Artificial Sequence
  cDNA sequence NM 003140.2
attttgtcgc

SEQ ID NO: 121 - DNA, 10 bp, Artificial Sequence
  cDNA sequence NM 003140.2
ttttgtcgca

SEQ ID NO: 122 - DNA, 10 bp, Artificial Sequence
  cDNA sequence NM 003140.2
tttgtcgcac

SEQ ID NO: 123 - DNA, 10 bp, Artificial Sequence
  cDNA sequence NM 003140.2
ttgtcgcact

SEQ ID NO: 124 - DNA, 10 bp, Artificial Sequence
  cDNA sequence NM 003140.2
tgtcgcactc

SEQ ID NO: 125 - DNA, 10 bp, Artificial Sequence
  cDNA sequence NM 003140.2
gtcgcactct

SEQ ID NO: 126 - DNA, 10 bp, Artificial Sequence
  cDNA sequence NM 003140.2
tcgcactctc

SEQ ID NO: 127 - DNA, 10 bp, Artificial Sequence
  cDNA sequence NM 003140.2
cgcactctcc

SEQ ID NO: 128 - DNA, 10 bp, Artificial Sequence
  cDNA sequence NM 003140.2
gcactctcct

SEQ ID NO: 129 - DNA, 10 bp, Artificial Sequence
  cDNA sequence NM 003140.2
cactctcctt

SEQ ID NO: 130 - DNA, 10 bp, Artificial Sequence
  cDNA sequence NM 003140.2
actctccttg

SEQ ID NO: 131 - DNA, 10 bp, Artificial Sequence
  cDNA sequence NM 003140.2
ctctccttgt

SEQ ID NO: 132 - DNA, 10 bp, Artificial Sequence
  cDNA sequence NM 003140.2
tctccttgtt

SEQ ID NO: 133 - DNA, 10 bp, Artificial Sequence
  cDNA sequence NM 003140.2
ctccttgttt

SEQ ID NO: 134 - DNA, 10 bp, Artificial Sequence
  cDNA sequence NM 003140.2
tccttgtttt

SEQ ID NO: 135 - DNA, 10 bp, Artificial Sequence
  cDNA sequence NM 003140.2
ccttgttttt

SEQ ID NO: 136 - DNA, 10 bp, Artificial Sequence
  cDNA sequence NM 003140.2
cttgtttttg

SEQ ID NO: 137 - DNA, 10 bp, Artificial Sequence
  cDNA sequence NM 003140.2
ttgtttttga

SEQ ID NO: 138 - DNA, 10 bp, Artificial Sequence
  cDNA sequence NM 003140.2
tgtttttgac

SEQ ID NO: 139 - DNA, 10 bp, Artificial Sequence
  cDNA sequence NM 003140.2
gtttttgaca

SEQ ID NO: 140 - DNA, 10 bp, Artificial Sequence
  cDNA sequence NM 003140.2
tttttgacaa

SEQ ID NO: 141 - DNA, 10 bp, Artificial Sequence
  cDNA sequence NM 003140.2
ttttgacaat

SEQ ID NO: 142 - DNA, 10 bp, Artificial Sequence
  cDNA sequence NM 003140.2
tttgacaatg

SEQ ID NO: 143 - DNA, 10 bp, Artificial Sequence
  cDNA sequence NM 003140.2
ttgacaatgc

SEQ ID NO: 144 - DNA, 10 bp, Artificial Sequence
  cDNA sequence NM 003140.2
tgacaatgca

SEQ ID NO: 145 - DNA, 10 bp, Artificial Sequence
  cDNA sequence NM 003140.2
gacaatgcaa

SEQ ID NO: 146 - DNA, 10 bp, Artificial Sequence
  cDNA sequence NM 003140.2
acaatgcaat

SEQ ID NO: 147 - DNA, 10 bp, Artificial Sequence
  cDNA sequence NM 003140.2
caatgcaatc

SEQ ID NO: 148 - DNA, 10 bp, Artificial Sequence
  cDNA sequence NM 003140.2
aatgcaatca

SEQ ID NO: 149 - DNA, 10 bp, Artificial Sequence
  cDNA sequence NM 003140.2
atgcaatcat

SEQ ID NO: 150 - DNA, 10 bp, Artificial Sequence
  cDNA sequence NM 003140.2
tgcaatcata

SEQ ID NO: 151 - DNA, 10 bp, Artificial Sequence
  cDNA sequence NM 003140.2
gcaatcatat

SEQ ID NO: 152 - DNA, 10 bp, Artificial Sequence
  cDNA sequence NM 003140.2
caatcatatg

SEQ ID NO: 153 - DNA, 10 bp, Artificial Sequence
  cDNA sequence NM 003140.2
aatcatatgc

SEQ ID NO: 154 - DNA, 10 bp, Artificial Sequence
  cDNA sequence NM 003140.2
atcatatgct

SEQ ID NO: 155 - DNA, 10 bp, Artificial Sequence
  cDNA sequence NM 003140.2
tcatatgctt

SEQ ID NO: 156 - DNA, 10 bp, Artificial Sequence
  cDNA sequence NM 003140.2
catatgcttc

SEQ ID NO: 157 - DNA, 10 bp, Artificial Sequence
  cDNA sequence NM 003140.2
atatgcttct

SEQ ID NO: 158 - DNA, 10 bp, Artificial Sequence
  cDNA sequence NM 003140.2
tatgcttctg

SEQ ID NO: 159 - DNA, 10 bp, Artificial Sequence
  cDNA sequence NM 003140.2
atgcttctgc

SEQ ID NO: 160 - DNA, 10 bp, Artificial Sequence
  cDNA sequence NM 003140.2
tgcttctgct

SEQ ID NO: 161 - DNA, 10 bp, Artificial Sequence
  cDNA sequence NM 003140.2
gcttctgcta

SEQ ID NO: 162 - DNA, 10 bp, Artificial Sequence
  cDNA sequence NM 003140.2
cttctgctat

SEQ ID NO: 163 - DNA, 10 bp, Artificial Sequence
  cDNA sequence NM 003140.2
ttctgctatg

SEQ ID NO: 164 - DNA, 10 bp, Artificial Sequence
  cDNA sequence NM 003140.2
tctgctatgt

SEQ ID NO: 165 - DNA, 10 bp, Artificial Sequence
  cDNA sequence NM 003140.2
ctgctatgtt

SEQ ID NO: 166 - DNA, 10 bp, Artificial Sequence
  cDNA sequence NM 003140.2
tgctatgtta

SEQ ID NO: 167 - DNA, 10 bp, Artificial Sequence
  cDNA sequence NM 003140.2
gctatgttaa

SEQ ID NO: 168 - DNA, 10 bp, Artificial Sequence
  cDNA sequence NM 003140.2
ctatgttaag

SEQ ID NO: 169 - DNA, 10 bp, Artificial Sequence
  cDNA sequence NM 003140.2
tatgttaagc

SEQ ID NO: 170 - DNA, 10 bp, Artificial Sequence
  cDNA sequence NM 003140.2
atgttaagcg

SEQ ID NO: 171 - DNA, 10 bp, Artificial Sequence
  cDNA sequence NM 003140.2
tgttaagcgt

SEQ ID NO: 172 - DNA, 10 bp, Artificial Sequence
  cDNA sequence NM 003140.2
gttaagcgta

SEQ ID NO: 173 - DNA, 10 bp, Artificial Sequence
  cDNA sequence NM 003140.2
ttaagcgtat

SEQ ID NO: 174 - DNA, 10 bp, Artificial Sequence
  cDNA sequence NM 003140.2
taagcgtatt

SEQ ID NO: 175 - DNA, 10 bp, Artificial Sequence
  cDNA sequence NM 003140.2
aagcgtattc

SEQ ID NO: 176 - DNA, 10 bp, Artificial Sequence
  cDNA sequence NM 003140.2
agcgtattca

SEQ ID NO: 177 - DNA, 10 bp, Artificial Sequence
  cDNA sequence NM 003140.2
gcgtattcaa

SEQ ID NO: 178 - DNA, 10 bp, Artificial Sequence
  cDNA sequence NM 003140.2
cgtattcaac

SEQ ID NO: 179 - DNA, 10 bp, Artificial Sequence
  cDNA sequence NM 003140.2
gtattcaaca

SEQ ID NO: 180 - DNA, 10 bp, Artificial Sequence
  cDNA sequence NM 003140.2
tattcaacag

SEQ ID NO: 181 - DNA, 10 bp, Artificial Sequence
  cDNA sequence NM 003140.2
attcaacagc

SEQ ID NO: 182 - DNA, 10 bp, Artificial Sequence
  cDNA sequence NM 003140.2
ttcaacagcg

SEQ ID NO: 183 - DNA, 10 bp, Artificial Sequence
  cDNA sequence NM 003140.2
tcaacagcga

SEQ ID NO: 184 - DNA, 10 bp, Artificial Sequence
  cDNA sequence NM 003140.2
caacagcgat

SEQ ID NO: 185 - DNA, 10 bp, Artificial Sequence
  cDNA sequence NM 003140.2
aacagcgatg

SEQ ID NO: 186 - DNA, 10 bp, Artificial Sequence
  cDNA sequence NM 003140.2
acagcgatga

SEQ ID NO: 187 - DNA, 10 bp, Artificial Sequence
  cDNA sequence NM 003140.2
cagcgatgat

SEQ ID NO: 188 - DNA, 10 bp, Artificial Sequence
  cDNA sequence NM 003140.2
agcgatgatt

SEQ ID NO: 189 - DNA, 10 bp, Artificial Sequence
  cDNA sequence NM 003140.2
gcgatgatta

SEQ ID NO: 190 - DNA, 10 bp, Artificial Sequence
  cDNA sequence NM 003140.2
cgatgattac

SEQ ID NO: 191 - DNA, 10 bp, Artificial Sequence
  cDNA sequence NM 003140.2
gatgattaca

SEQ ID NO: 192 - DNA, 10 bp, Artificial Sequence
  cDNA sequence NM 003140.2
atgattacag

SEQ ID NO: 193 - DNA, 10 bp, Artificial Sequence
  cDNA sequence NM 003140.2
tgattacagt

SEQ ID NO: 194 - DNA, 10 bp, Artificial Sequence
  cDNA sequence NM 003140.2
gattacagtc

SEQ ID NO: 195 - DNA, 10 bp, Artificial Sequence
  cDNA sequence NM 003140.2
attacagtcc

SEQ ID NO: 196 - DNA, 10 bp, Artificial Sequence
  cDNA sequence NM 003140.2
ttacagtcca

SEQ ID NO: 197 - DNA, 10 bp, Artificial Sequence
  cDNA sequence NM 003140.2
tacagtccag

SEQ ID NO: 198 - DNA, 10 bp, Artificial Sequence
  cDNA sequence NM 003140.2
acagtccagc

SEQ ID NO: 199 - DNA, 10 bp, Artificial Sequence
  cDNA sequence NM 003140.2
cagtccagct

SEQ ID NO: 200 - DNA, 10 bp, Artificial Sequence
  cDNA sequence NM 003140.2
agtccagctg

SEQ ID NO: 201 - DNA, 10 bp, Artificial Sequence
  cDNA sequence NM 003140.2
gtccagctgt

SEQ ID NO: 202 - DNA, 10 bp, Artificial Sequence
  cDNA sequence NM 003140.2
tccagctgtg

SEQ ID NO: 203 - DNA, 10 bp, Artificial Sequence
  cDNA sequence NM 003140.2
ccagctgtgc

SEQ ID NO: 204 - DNA, 10 bp, Artificial Sequence
  cDNA sequence NM 003140.2
cagctgtgca

SEQ ID NO: 205 - DNA, 10 bp, Artificial Sequence
  cDNA sequence NM 003140.2
agctgtgcaa

SEQ ID NO: 206 - DNA, 10 bp, Artificial Sequence
  cDNA sequence NM 003140.2
gctgtgcaag

SEQ ID NO: 207 - DNA, 10 bp, Artificial Sequence
  cDNA sequence NM 003140.2
ctgtgcaaga

SEQ ID NO: 208 - DNA, 10 bp, Artificial Sequence
  cDNA sequence NM 003140.2
tgtgcaagag

SEQ ID NO: 209 - DNA, 10 bp, Artificial Sequence
  cDNA sequence NM 003140.2
gtgcaagaga

SEQ ID NO: 210 - DNA, 10 bp, Artificial Sequence
  cDNA sequence NM 003140.2
tgcaagagaa

SEQ ID NO: 211 - DNA, 10 bp, Artificial Sequence
  cDNA sequence NM 003140.2
gcaagagaat

SEQ ID NO: 212 - DNA, 10 bp, Artificial Sequence
  cDNA sequence NM 003140.2
caagagaata

SEQ ID NO: 213 - DNA, 10 bp, Artificial Sequence
  cDNA sequence NM 003140.2
aagagaatat

SEQ ID NO: 214 - DNA, 10 bp, Artificial Sequence
  cDNA sequence NM 003140.2
agagaatatt

SEQ ID NO: 215 - DNA, 10 bp, Artificial Sequence
  cDNA sequence NM 003140.2
gagaatattc

SEQ ID NO: 216 - DNA, 10 bp, Artificial Sequence
  cDNA sequence NM 003140.2
agaatattcc

SEQ ID NO: 217 - DNA, 10 bp, Artificial Sequence
  cDNA sequence NM 003140.2
gaatattccc

SEQ ID NO: 218 - DNA, 10 bp, Artificial Sequence
  cDNA sequence NM 003140.2
aatattcccg

SEQ ID NO: 219 - DNA, 10 bp, Artificial Sequence
  cDNA sequence NM 003140.2
atattcccgc

SEQ ID NO: 220 - DNA, 10 bp, Artificial Sequence
  cDNA sequence NM 003140.2
tattcccgct

SEQ ID NO: 221 - DNA, 10 bp, Artificial Sequence
  cDNA sequence NM 003140.2
attcccgctc

SEQ ID NO: 222 - DNA, 10 bp, Artificial Sequence
  cDNA sequence NM 003140.2
ttcccgctct

SEQ ID NO: 223 - DNA, 10 bp, Artificial Sequence
  cDNA sequence NM 003140.2
tcccgctctc

SEQ ID NO: 224 - DNA, 10 bp, Artificial Sequence
  cDNA sequence NM 003140.2
cccgctctcc

SEQ ID NO: 225 - DNA, 10 bp, Artificial Sequence
  cDNA sequence NM 003140.2
ccgctctccg

SEQ ID NO: 226 - DNA, 10 bp, Artificial Sequence
  cDNA sequence NM 003140.2
cgctctccgg

SEQ ID NO: 227 - DNA, 10 bp, Artificial Sequence
  cDNA sequence NM 003140.2
gctctccgga

SEQ ID NO: 228 - DNA, 10 bp, Artificial Sequence
  cDNA sequence NM 003140.2
ctctccggag

SEQ ID NO: 229 - DNA, 10 bp, Artificial Sequence
  cDNA sequence NM 003140.2
tctccggaga

SEQ ID NO: 230 - DNA, 10 bp, Artificial Sequence
  cDNA sequence NM 003140.2
ctccggagaa

SEQ ID NO: 231 - DNA, 10 bp, Artificial Sequence
  cDNA sequence NM 003140.2
tccggagaag

SEQ ID NO: 232 - DNA, 10 bp, Artificial Sequence
  cDNA sequence NM 003140.2
ccggagaagc

SEQ ID NO: 233 - DNA, 10 bp, Artificial Sequence
  cDNA sequence NM 003140.2
cggagaagct

SEQ ID NO: 234 - DNA, 10 bp, Artificial Sequence
  cDNA sequence NM 003140.2
ggagaagctc

SEQ ID NO: 235 - DNA, 10 bp, Artificial Sequence
  cDNA sequence NM 003140.2
gagaagctct

SEQ ID NO: 236 - DNA, 10 bp, Artificial Sequence
  cDNA sequence NM 003140.2
agaagctctt

SEQ ID NO: 237 - DNA, 10 bp, Artificial Sequence
  cDNA sequence NM 003140.2
gaagctcttc

SEQ ID NO: 238 - DNA, 10 bp, Artificial Sequence
  cDNA sequence NM 003140.2
aagctcttcc

SEQ ID NO: 239 - DNA, 10 bp, Artificial Sequence
  cDNA sequence NM 003140.2
agctcttcct

SEQ ID NO: 240 - DNA, 10 bp, Artificial Sequence
  cDNA sequence NM 003140.2
gctcttcctt

SEQ ID NO: 241 - DNA, 10 bp, Artificial Sequence
  cDNA sequence NM 003140.2
ctcttccttc

SEQ ID NO: 242 - DNA, 10 bp, Artificial Sequence
  cDNA sequence NM 003140.2
tcttccttcc

SEQ ID NO: 243 - DNA, 10 bp, Artificial Sequence
  cDNA sequence NM 003140.2
cttccttcct

SEQ ID NO: 244 - DNA, 10 bp, Artificial Sequence
  cDNA sequence NM 003140.2
ttccttcctt

SEQ ID NO: 245 - DNA, 10 bp, Artificial Sequence
  cDNA sequence NM 003140.2
tccttccttt

SEQ ID NO: 246 - DNA, 10 bp, Artificial Sequence
  cDNA sequence NM 003140.2
ccttcctttg

SEQ ID NO: 247 - DNA, 10 bp, Artificial Sequence
  cDNA sequence NM 003140.2
cttcctttgc

SEQ ID NO: 248 - DNA, 10 bp, Artificial Sequence
  cDNA sequence NM 003140.2
ttcctttgca

SEQ ID NO: 249 - DNA, 10 bp, Artificial Sequence
  cDNA sequence NM 003140.2
tcctttgcac

SEQ ID NO: 250 - DNA, 10 bp, Artificial Sequence
  cDNA sequence NM 003140.2
cctttgcact

SEQ ID NO: 251 - DNA, 10 bp, Artificial Sequence
  cDNA sequence NM 003140.2
ctttgcactg

SEQ ID NO: 252 - DNA, 10 bp, Artificial Sequence
  cDNA sequence NM 003140.2
tttgcactga

SEQ ID NO: 253 - DNA, 10 bp, Artificial Sequence
  cDNA sequence NM 003140.2
ttgcactgaa

SEQ ID NO: 254 - DNA, 10 bp, Artificial Sequence
  cDNA sequence NM 003140.2
tgcactgaaa

SEQ ID NO: 255 - DNA, 10 bp, Artificial Sequence
  cDNA sequence NM 003140.2
gcactgaaag

SEQ ID NO: 256 - DNA, 10 bp, Artificial Sequence
  cDNA sequence NM 003140.2
cactgaaagc

SEQ ID NO: 257 - DNA, 10 bp, Artificial Sequence
  cDNA sequence NM 003140.2
actgaaagct

SEQ ID NO: 258 - DNA, 10 bp, Artificial Sequence
  cDNA sequence NM 003140.2
ctgaaagctg

SEQ ID NO: 259 - DNA, 10 bp, Artificial Sequence
  cDNA sequence NM 003140.2
tgaaagctgt

SEQ ID NO: 260 - DNA, 10 bp, Artificial Sequence
  cDNA sequence NM 003140.2
gaaagctgta

SEQ ID NO: 261 - DNA, 10 bp, Artificial Sequence
  cDNA sequence NM 003140.2
aaagctgtaa

SEQ ID NO: 262 - DNA, 10 bp, Artificial Sequence
  cDNA sequence NM 003140.2
aagctgtaac

SEQ ID NO: 263 - DNA, 10 bp, Artificial Sequence
  cDNA sequence NM 003140.2
agctgtaact

SEQ ID NO: 264 - DNA, 10 bp, Artificial Sequence
  cDNA sequence NM 003140.2
gctgtaactc

SEQ ID NO: 265 - DNA, 10 bp, Artificial Sequence
  cDNA sequence NM 003140.2
ctgtaactct

SEQ ID NO: 266 - DNA, 10 bp, Artificial Sequence
  cDNA sequence NM 003140.2
tgtaactcta

SEQ ID NO: 267 - DNA, 10 bp, Artificial Sequence
  cDNA sequence NM 003140.2
gtaactctaa

SEQ ID NO: 268 - DNA, 10 bp, Artificial Sequence
  cDNA sequence NM 003140.2
taactctaag

SEQ ID NO: 269 - DNA, 10 bp, Artificial Sequence
  cDNA sequence NM 003140.2
aactctaagt

SEQ ID NO: 270 - DNA, 10 bp, Artificial Sequence
  cDNA sequence NM 003140.2
actctaagta

SEQ ID NO: 271 - DNA, 10 bp, Artificial Sequence
  cDNA sequence NM 003140.2
ctctaagtat

SEQ ID NO: 272 - DNA, 10 bp, Artificial Sequence
  cDNA sequence NM 003140.2
tctaagtatc

SEQ ID NO: 273 - DNA, 10 bp, Artificial Sequence
  cDNA sequence NM 003140.2
ctaagtatca

SEQ ID NO: 274 - DNA, 10 bp, Artificial Sequence
  cDNA sequence NM 003140.2
taagtatcag

SEQ ID NO: 275 - DNA, 10 bp, Artificial Sequence
  cDNA sequence NM 003140.2
aagtatcagt

SEQ ID NO: 276 - DNA, 10 bp, Artificial Sequence
  cDNA sequence NM 003140.2
agtatcagtg

SEQ ID NO: 277 - DNA, 10 bp, Artificial Sequence
  cDNA sequence NM 003140.2
gtatcagtgt

SEQ ID NO: 278 - DNA, 10 bp, Artificial Sequence
  cDNA sequence NM 003140.2
tatcagtgtg

SEQ ID NO: 279 - DNA, 10 bp, Artificial Sequence
  cDNA sequence NM 003140.2
atcagtgtga

SEQ ID NO: 280 - DNA, 10 bp, Artificial Sequence
  cDNA sequence NM 003140.2
tcagtgtgaa

SEQ ID NO: 281 - DNA, 10 bp, Artificial Sequence
  cDNA sequence NM 003140.2
cagtgtgaaa

SEQ ID NO: 282 - DNA, 10 bp, Artificial Sequence
  cDNA sequence NM 003140.2
agtgtgaaac

SEQ ID NO: 283 - DNA, 10 bp, Artificial Sequence
  cDNA sequence NM 003140.2
gtgtgaaacg

SEQ ID NO: 284 - DNA, 10 bp, Artificial Sequence
  cDNA sequence NM 003140.2
tgtgaaacgg

SEQ ID NO: 285 - DNA, 10 bp, Artificial Sequence
  cDNA sequence NM 003140.2
gtgaaacggg

SEQ ID NO: 286 - DNA, 10 bp, Artificial Sequence
  cDNA sequence NM 003140.2
tgaaacggga

SEQ ID NO: 287 - DNA, 10 bp, Artificial Sequence
  cDNA sequence NM 003140.2
gaaacgggag

SEQ ID NO: 288 - DNA, 10 bp, Artificial Sequence
  cDNA sequence NM 003140.2
aaacgggaga

SEQ ID NO: 289 - DNA, 10 bp, Artificial Sequence
  cDNA sequence NM 003140.2
aacgggagaa

SEQ ID NO: 290 - DNA, 10 bp, Artificial Sequence
  cDNA sequence NM 003140.2
acgggagaaa

SEQ ID NO: 291 - DNA, 10 bp, Artificial Sequence
  cDNA sequence NM 003140.2
cgggagaaaa

SEQ ID NO: 292 - DNA, 10 bp, Artificial Sequence
  cDNA sequence NM 003140.2
gggagaaaac

SEQ ID NO: 293 - DNA, 10 bp, Artificial Sequence
  cDNA sequence NM 003140.2
ggagaaaaca

SEQ ID NO: 294 - DNA, 10 bp, Artificial Sequence
  cDNA sequence NM 003140.2
gagaaaacag

SEQ ID NO: 295 - DNA, 10 bp, Artificial Sequence
  cDNA sequence NM 003140.2
agaaaacagt

SEQ ID NO: 296 - DNA, 10 bp, Artificial Sequence
  cDNA sequence NM 003140.2
gaaaacagta

SEQ ID NO: 297 - DNA, 10 bp, Artificial Sequence
  cDNA sequence NM 003140.2
aaaacagtaa

SEQ ID NO: 298 - DNA, 10 bp, Artificial Sequence
  cDNA sequence NM 003140.2
aaacagtaaa

SEQ ID NO: 299 - DNA, 10 bp, Artificial Sequence
  cDNA sequence NM 003140.2
aacagtaaag

SEQ ID NO: 300 - DNA, 10 bp, Artificial Sequence
  cDNA sequence NM 003140.2
acagtaaagg

SEQ ID NO: 301 - DNA, 10 bp, Artificial Sequence
  cDNA sequence NM 003140.2
cagtaaaggc

SEQ ID NO: 302 - DNA, 10 bp, Artificial Sequence
  cDNA sequence NM 003140.2
agtaaaggca

SEQ ID NO: 303 - DNA, 10 bp, Artificial Sequence
  cDNA sequence NM 003140.2
gtaaaggcaa

SEQ ID NO: 304 - DNA, 10 bp, Artificial Sequence
  cDNA sequence NM 003140.2
taaaggcaac

SEQ ID NO: 305 - DNA, 10 bp, Artificial Sequence
  cDNA sequence NM 003140.2
aaaggcaacg

SEQ ID NO: 306 - DNA, 10 bp, Artificial Sequence
  cDNA sequence NM 003140.2
aaggcaacgt

SEQ ID NO: 307 - DNA, 10 bp, Artificial Sequence
  cDNA sequence NM 003140.2
aggcaacgtc

SEQ ID NO: 308 - DNA, 10 bp, Artificial Sequence
  cDNA sequence NM 003140.2
ggcaacgtcc

SEQ ID NO: 309 - DNA, 10 bp, Artificial Sequence
  cDNA sequence NM 003140.2
gcaacgtcca

SEQ ID NO: 310 - DNA, 10 bp, Artificial Sequence
  cDNA sequence NM 003140.2
caacgtccag

SEQ ID NO: 311 - DNA, 10 bp, Artificial Sequence
  cDNA sequence NM 003140.2
aacgtccagg

SEQ ID NO: 312 - DNA, 10 bp, Artificial Sequence
  cDNA sequence NM 003140.2
acgtccagga

SEQ ID NO: 313 - DNA, 10 bp, Artificial Sequence
  cDNA sequence NM 003140.2
cgtccaggat

SEQ ID NO: 314 - DNA, 10 bp, Artificial Sequence
  cDNA sequence NM 003140.2
gtccaggata

SEQ ID NO: 315 - DNA, 10 bp, Artificial Sequence
  cDNA sequence NM 003140.2
tccaggatag

SEQ ID NO: 316 - DNA, 10 bp, Artificial Sequence
  cDNA sequence NM 003140.2
ccaggataga

SEQ ID NO: 317 - DNA, 10 bp, Artificial Sequence
  cDNA sequence NM 003140.2
caggatagag

SEQ ID NO: 318 - DNA, 10 bp, Artificial Sequence
  cDNA sequence NM 003140.2
aggatagagt

SEQ ID NO: 319 - DNA, 10 bp, Artificial Sequence
  cDNA sequence NM 003140.2
ggatagagtg

SEQ ID NO: 320 - DNA, 10 bp, Artificial Sequence
  cDNA sequence NM 003140.2
gatagagtga

SEQ ID NO: 321 - DNA, 10 bp, Artificial Sequence
  cDNA sequence NM 003140.2
atagagtgaa

SEQ ID NO: 322 - DNA, 10 bp, Artificial Sequence
  cDNA sequence NM 003140.2
tagagtgaag

SEQ ID NO: 323 - DNA, 10 bp, Artificial Sequence
  cDNA sequence NM 003140.2
agagtgaagc

SEQ ID NO: 324 - DNA, 10 bp, Artificial Sequence
  cDNA sequence NM 003140.2
gagtgaagcg

SEQ ID NO: 325 - DNA, 10 bp, Artificial Sequence
  cDNA sequence NM 003140.2
agtgaagcga

SEQ ID NO: 326 - DNA, 10 bp, Artificial Sequence
  cDNA sequence NM 003140.2
gtgaagcgac

SEQ ID NO: 327 - DNA, 10 bp, Artificial Sequence
  cDNA sequence NM 003140.2
tgaagcgacc

SEQ ID NO: 328 - DNA, 10 bp, Artificial Sequence
  cDNA sequence NM 003140.2
gaagcgaccc

SEQ ID NO: 329 - DNA, 10 bp, Artificial Sequence
  cDNA sequence NM 003140.2
aagcgaccca

SEQ ID NO: 330 - DNA, 10 bp, Artificial Sequence
  cDNA sequence NM 003140.2
agcgacccat

SEQ ID NO: 331 - DNA, 10 bp, Artificial Sequence
  cDNA sequence NM 003140.2
gcgacccatg

SEQ ID NO: 332 - DNA, 10 bp, Artificial Sequence
  cDNA sequence NM 003140.2
cgacccatga

SEQ ID NO: 333 - DNA, 10 bp, Artificial Sequence
  cDNA sequence NM 003140.2
gacccatgaa

SEQ ID NO: 334 - DNA, 10 bp, Artificial Sequence
  cDNA sequence NM 003140.2
acccatgaac

SEQ ID NO: 335 - DNA, 10 bp, Artificial Sequence
  cDNA sequence NM 003140.2
cccatgaacg

SEQ ID NO: 336 - DNA, 10 bp, Artificial Sequence
  cDNA sequence NM 003140.2
ccatgaacgc

SEQ ID NO: 337 - DNA, 10 bp, Artificial Sequence
  cDNA sequence NM 003140.2
catgaacgca

SEQ ID NO: 338 - DNA, 10 bp, Artificial Sequence
  cDNA sequence NM 003140.2
atgaacgcat

SEQ ID NO: 339 - DNA, 10 bp, Artificial Sequence
  cDNA sequence NM 003140.2
tgaacgcatt

SEQ ID NO: 340 - DNA, 10 bp, Artificial Sequence
  cDNA sequence NM 003140.2
gaacgcattc

SEQ ID NO: 341 - DNA, 10 bp, Artificial Sequence
  cDNA sequence NM 003140.2
aacgcattca

SEQ ID NO: 342 - DNA, 10 bp, Artificial Sequence
  cDNA sequence NM 003140.2
acgcattcat

SEQ ID NO: 343 - DNA, 10 bp, Artificial Sequence
  cDNA sequence NM 003140.2
cgcattcatc

SEQ ID NO: 344 - DNA, 10 bp, Artificial Sequence
  cDNA sequence NM 003140.2
gcattcatcg

SEQ ID NO: 345 - DNA, 10 bp, Artificial Sequence
  cDNA sequence NM 003140.2
cattcatcgt

SEQ ID NO: 346 - DNA, 10 bp, Artificial Sequence
  cDNA sequence NM 003140.2
attcatcgtg

SEQ ID NO: 347 - DNA, 10 bp, Artificial Sequence
  cDNA sequence NM 003140.2
ttcatcgtgt

SEQ ID NO: 348 - DNA, 10 bp, Artificial Sequence
  cDNA sequence NM 003140.2
tcatcgtgtg

SEQ ID NO: 349 - DNA, 10 bp, Artificial Sequence
  cDNA sequence NM 003140.2
catcgtgtgg

SEQ ID NO: 350 - DNA, 10 bp, Artificial Sequence
  cDNA sequence NM 003140.2
atcgtgtggt

SEQ ID NO: 351 - DNA, 10 bp, Artificial Sequence
  cDNA sequence NM 003140.2
tcgtgtggtc

SEQ ID NO: 352 - DNA, 10 bp, Artificial Sequence
  cDNA sequence NM 003140.2
cgtgtggtct

SEQ ID NO: 353 - DNA, 10 bp, Artificial Sequence
  cDNA sequence NM 003140.2
gtgtggtctc

SEQ ID NO: 354 - DNA, 10 bp, Artificial Sequence
  cDNA sequence NM 003140.2
tgtggtctcg

SEQ ID NO: 355 - DNA, 10 bp, Artificial Sequence
  cDNA sequence NM 003140.2
gtggtctcgc

SEQ ID NO: 356 - DNA, 10 bp, Artificial Sequence
  cDNA sequence NM 003140.2
tggtctcgcg

SEQ ID NO: 357 - DNA, 10 bp, Artificial Sequence
  cDNA sequence NM 003140.2
ggtctcgcga

SEQ ID NO: 358 - DNA, 10 bp, Artificial Sequence
  cDNA sequence NM 003140.2
gtctcgcgat

SEQ ID NO: 359 - DNA, 10 bp, Artificial Sequence
  cDNA sequence NM 003140.2
tctcgcgatc

SEQ ID NO: 360 - DNA, 10 bp, Artificial Sequence
  cDNA sequence NM 003140.2
ctcgcgatca

SEQ ID NO: 361 - DNA, 10 bp, Artificial Sequence
  cDNA sequence NM 003140.2
tcgcgatcag

SEQ ID NO: 362 - DNA, 10 bp, Artificial Sequence
  cDNA sequence NM 003140.2
cgcgatcaga

SEQ ID NO: 363 - DNA, 10 bp, Artificial Sequence
  cDNA sequence NM 003140.2
gcgatcagag

SEQ ID NO: 364 - DNA, 10 bp, Artificial Sequence
  cDNA sequence NM 003140.2
cgatcagagg

SEQ ID NO: 365 - DNA, 10 bp, Artificial Sequence
  cDNA sequence NM 003140.2
gatcagaggc

SEQ ID NO: 366 - DNA, 10 bp, Artificial Sequence
  cDNA sequence NM 003140.2
atcagaggcg

SEQ ID NO: 367 - DNA, 10 bp, Artificial Sequence
  cDNA sequence NM 003140.2
tcagaggcgc

SEQ ID NO: 368 - DNA, 10 bp, Artificial Sequence
  cDNA sequence NM 003140.2
cagaggcgca

SEQ ID NO: 369 - DNA, 10 bp, Artificial Sequence
  cDNA sequence NM 003140.2
agaggcgcaa

SEQ ID NO: 370 - DNA, 10 bp, Artificial Sequence
  cDNA sequence NM 003140.2
gaggcgcaag

SEQ ID NO: 371 - DNA, 10 bp, Artificial Sequence
  cDNA sequence NM 003140.2
aggcgcaaga

SEQ ID NO: 372 - DNA, 10 bp, Artificial Sequence
  cDNA sequence NM 003140.2
ggcgcaagat

SEQ ID NO: 373 - DNA, 10 bp, Artificial Sequence
  cDNA sequence NM 003140.2
gcgcaagatg

SEQ ID NO: 374 - DNA, 10 bp, Artificial Sequence
  cDNA sequence NM 003140.2
cgcaagatgg

SEQ ID NO: 375 - DNA, 10 bp, Artificial Sequence
  cDNA sequence NM 003140.2
gcaagatggc

SEQ ID NO: 376 - DNA, 10 bp, Artificial Sequence
  cDNA sequence NM 003140.2
caagatggct

SEQ ID NO: 377 - DNA, 10 bp, Artificial Sequence
  cDNA sequence NM 003140.2
aagatggctc

SEQ ID NO: 378 - DNA, 10 bp, Artificial Sequence
  cDNA sequence NM 003140.2
agatggctct

SEQ ID NO: 379 - DNA, 10 bp, Artificial Sequence
  cDNA sequence NM 003140.2
gatggctcta

SEQ ID NO: 380 - DNA, 10 bp, Artificial Sequence
  cDNA sequence NM 003140.2
atggctctag

SEQ ID NO: 381 - DNA, 10 bp, Artificial Sequence
  cDNA sequence NM 003140.2
tggctctaga

SEQ ID NO: 382 - DNA, 10 bp, Artificial Sequence
  cDNA sequence NM 003140.2
ggctctagag

SEQ ID NO: 383 - DNA, 10 bp, Artificial Sequence
  cDNA sequence NM 003140.2
gctctagaga

SEQ ID NO: 384 - DNA, 10 bp, Artificial Sequence
  cDNA sequence NM 003140.2
ctctagagaa

SEQ ID NO: 385 - DNA, 10 bp, Artificial Sequence
  cDNA sequence NM 003140.2
tctagagaat

SEQ ID NO: 386 - DNA, 10 bp, Artificial Sequence
  cDNA sequence NM 003140.2
ctagagaatc

SEQ ID NO: 387 - DNA, 10 bp, Artificial Sequence
  cDNA sequence NM 003140.2
tagagaatcc

SEQ ID NO: 388 - DNA, 10 bp, Artificial Sequence
  cDNA sequence NM 003140.2
agagaatccc

SEQ ID NO: 389 - DNA, 10 bp, Artificial Sequence
  cDNA sequence NM 003140.2
gagaatccca

SEQ ID NO: 390 - DNA, 10 bp, Artificial Sequence
  cDNA sequence NM 003140.2
agaatcccag

SEQ ID NO: 391 - DNA, 10 bp, Artificial Sequence
  cDNA sequence NM 003140.2
gaatcccaga

SEQ ID NO: 392 - DNA, 10 bp, Artificial Sequence
  cDNA sequence NM 003140.2
aatcccagaa

SEQ ID NO: 393 - DNA, 10 bp, Artificial Sequence
  cDNA sequence NM 003140.2
atcccagaat

SEQ ID NO: 394 - DNA, 10 bp, Artificial Sequence
  cDNA sequence NM 003140.2
tcccagaatg

SEQ ID NO: 395 - DNA, 10 bp, Artificial Sequence
  cDNA sequence NM 003140.2
cccagaatgc

SEQ ID NO: 396 - DNA, 10 bp, Artificial Sequence
  cDNA sequence NM 003140.2
ccagaatgcg

SEQ ID NO: 397 - DNA, 10 bp, Artificial Sequence
  cDNA sequence NM 003140.2
cagaatgcga

SEQ ID NO: 398 - DNA, 10 bp, Artificial Sequence
  cDNA sequence NM 003140.2
agaatgcgaa

SEQ ID NO: 399 - DNA, 10 bp, Artificial Sequence
  cDNA sequence NM 003140.2
gaatgcgaaa

SEQ ID NO: 400 - DNA, 10 bp, Artificial Sequence
  cDNA sequence NM 003140.2
aatgcgaaac

SEQ ID NO: 401 - DNA, 10 bp, Artificial Sequence
  cDNA sequence NM 003140.2
atgcgaaact

SEQ ID NO: 402 - DNA, 10 bp, Artificial Sequence
  cDNA sequence NM 003140.2
tgcgaaactc

SEQ ID NO: 403 - DNA, 10 bp, Artificial Sequence
  cDNA sequence NM 003140.2
gcgaaactca

SEQ ID NO: 404 - DNA, 10 bp, Artificial Sequence
  cDNA sequence NM 003140.2
cgaaactcag

SEQ ID NO: 405 - DNA, 10 bp, Artificial Sequence
  cDNA sequence NM 003140.2
gaaactcaga

SEQ ID NO: 406 - DNA, 10 bp, Artificial Sequence
  cDNA sequence NM 003140.2
aaactcagag

SEQ ID NO: 407 - DNA, 10 bp, Artificial Sequence
  cDNA sequence NM 003140.2
aactcagaga

SEQ ID NO: 408 - DNA, 10 bp, Artificial Sequence
  cDNA sequence NM 003140.2
actcagagat

SEQ ID NO: 409 - DNA, 10 bp, Artificial Sequence
  cDNA sequence NM 003140.2
ctcagagatc

SEQ ID NO: 410 - DNA, 10 bp, Artificial Sequence
  cDNA sequence NM 003140.2
tcagagatca

SEQ ID NO: 411 - DNA, 10 bp, Artificial Sequence
  cDNA sequence NM 003140.2
cagagatcag

SEQ ID NO: 412 - DNA, 10 bp, Artificial Sequence
  cDNA sequence NM 003140.2
agagatcagc

SEQ ID NO: 413 - DNA, 10 bp, Artificial Sequence
  cDNA sequence NM 003140.2
gagatcagca

SEQ ID NO: 414 - DNA, 10 bp, Artificial Sequence
  cDNA sequence NM 003140.2
agatcagcaa

SEQ ID NO: 415 - DNA, 10 bp, Artificial Sequence
  cDNA sequence NM 003140.2
gatcagcaag

SEQ ID NO: 416 - DNA, 10 bp, Artificial Sequence
  cDNA sequence NM 003140.2
atcagcaagc

SEQ ID NO: 417 - DNA, 10 bp, Artificial Sequence
  cDNA sequence NM 003140.2
tcagcaagca

SEQ ID NO: 418 - DNA, 10 bp, Artificial Sequence
  cDNA sequence NM 003140.2
cagcaagcag

SEQ ID NO: 419 - DNA, 10 bp, Artificial Sequence
  cDNA sequence NM 003140.2
agcaagcagc

SEQ ID NO: 420 - DNA, 10 bp, Artificial Sequence
  cDNA sequence NM 003140.2
gcaagcagct

SEQ ID NO: 421 - DNA, 10 bp, Artificial Sequence
  cDNA sequence NM 003140.2
caagcagctg

SEQ ID NO: 422 - DNA, 10 bp, Artificial Sequence
  cDNA sequence NM 003140.2
aagcagctgg

SEQ ID NO: 423 - DNA, 10 bp, Artificial Sequence
  cDNA sequence NM 003140.2
agcagctggg

SEQ ID NO: 424 - DNA, 10 bp, Artificial Sequence
  cDNA sequence NM 003140.2
gcagctggga

SEQ ID NO: 425 - DNA, 10 bp, Artificial Sequence
  cDNA sequence NM 003140.2
cagctgggat

SEQ ID NO: 426 - DNA, 10 bp, Artificial Sequence
  cDNA sequence NM 003140.2
agctgggata

SEQ ID NO: 427 - DNA, 10 bp, Artificial Sequence
  cDNA sequence NM 003140.2
gctgggatac

SEQ ID NO: 428 - DNA, 10 bp, Artificial Sequence
  cDNA sequence NM 003140.2
ctgggatacc

SEQ ID NO: 429 - DNA, 10 bp, Artificial Sequence
  cDNA sequence NM 003140.2
tgggatacca

SEQ ID NO: 430 - DNA, 10 bp, Artificial Sequence
  cDNA sequence NM 003140.2
gggataccag

SEQ ID NO: 431 - DNA, 10 bp, Artificial Sequence
  cDNA sequence NM 003140.2
ggataccagt

SEQ ID NO: 432 - DNA, 10 bp, Artificial Sequence
  cDNA sequence NM 003140.2
gataccagtg

SEQ ID NO: 433 - DNA, 10 bp, Artificial Sequence
  cDNA sequence NM 003140.2
ataccagtgg

SEQ ID NO: 434 - DNA, 10 bp, Artificial Sequence
  cDNA sequence NM 003140.2
taccagtgga

SEQ ID NO: 435 - DNA, 10 bp, Artificial Sequence
  cDNA sequence NM 003140.2
accagtggaa

SEQ ID NO: 436 - DNA, 10 bp, Artificial Sequence
  cDNA sequence NM 003140.2
ccagtggaaa

SEQ ID NO: 437 - DNA, 10 bp, Artificial Sequence
  cDNA sequence NM 003140.2
cagtggaaaa

SEQ ID NO: 438 - DNA, 10 bp, Artificial Sequence
  cDNA sequence NM 003140.2
agtggaaaat

SEQ ID NO: 439 - DNA, 10 bp, Artificial Sequence
  cDNA sequence NM 003140.2
gtggaaaatg

SEQ ID NO: 440 - DNA, 10 bp, Artificial Sequence
  cDNA sequence NM 003140.2
tggaaaatgc

SEQ ID NO: 441 - DNA, 10 bp, Artificial Sequence
  cDNA sequence NM 003140.2
ggaaaatgct

SEQ ID NO: 442 - DNA, 10 bp, Artificial Sequence
  cDNA sequence NM 003140.2
gaaaatgctt

SEQ ID NO: 443 - DNA, 10 bp, Artificial Sequence
  cDNA sequence NM 003140.2
aaaatgctta

SEQ ID NO: 444 - DNA, 10 bp, Artificial Sequence
  cDNA sequence NM 003140.2
aaatgcttac

SEQ ID NO: 445 - DNA, 10 bp, Artificial Sequence
  cDNA sequence NM 003140.2
aatgcttact

SEQ ID NO: 446 - DNA, 10 bp, Artificial Sequence
  cDNA sequence NM 003140.2
atgcttactg

SEQ ID NO: 447 - DNA, 10 bp, Artificial Sequence
  cDNA sequence NM 003140.2
tgcttactga

SEQ ID NO: 448 - DNA, 10 bp, Artificial Sequence
  cDNA sequence NM 003140.2
gcttactgaa

SEQ ID NO: 449 - DNA, 10 bp, Artificial Sequence
  cDNA sequence NM 003140.2
cttactgaag

SEQ ID NO: 450 - DNA, 10 bp, Artificial Sequence
  cDNA sequence NM 003140.2
ttactgaagc

SEQ ID NO: 451 - DNA, 10 bp, Artificial Sequence
  cDNA sequence NM 003140.2
tactgaagcc

SEQ ID NO: 452 - DNA, 10 bp, Artificial Sequence
  cDNA sequence NM 003140.2
actgaagccg

SEQ ID NO: 453 - DNA, 10 bp, Artificial Sequence
  cDNA sequence NM 003140.2
ctgaagccga

SEQ ID NO: 454 - DNA, 10 bp, Artificial Sequence
  cDNA sequence NM 003140.2
tgaagccgaa

SEQ ID NO: 455 - DNA, 10 bp, Artificial Sequence
  cDNA sequence NM 003140.2
gaagccgaaa

SEQ ID NO: 456 - DNA, 10 bp, Artificial Sequence
  cDNA sequence NM 003140.2
aagccgaaaa

SEQ ID NO: 457 - DNA, 10 bp, Artificial Sequence
  cDNA sequence NM 003140.2
agccgaaaaa

SEQ ID NO: 458 - DNA, 10 bp, Artificial Sequence
  cDNA sequence NM 003140.2
gccgaaaaat

SEQ ID NO: 459 - DNA, 10 bp, Artificial Sequence
  cDNA sequence NM 003140.2
ccgaaaaatg

SEQ ID NO: 460 - DNA, 10 bp, Artificial Sequence
  cDNA sequence NM 003140.2
cgaaaaatgg

SEQ ID NO: 461 - DNA, 10 bp, Artificial Sequence
  cDNA sequence NM 003140.2
gaaaaatggc

SEQ ID NO: 462 - DNA, 10 bp, Artificial Sequence
  cDNA sequence NM 003140.2
aaaaatggcc

SEQ ID NO: 463 - DNA, 10 bp, Artificial Sequence
  cDNA sequence NM 003140.2
aaaatggcca

SEQ ID NO: 464 - DNA, 10 bp, Artificial Sequence
  cDNA sequence NM 003140.2
aaatggccat

SEQ ID NO: 465 - DNA, 10 bp, Artificial Sequence
  cDNA sequence NM 003140.2
aatggccatt

SEQ ID NO: 466 - DNA, 10 bp, Artificial Sequence
  cDNA sequence NM 003140.2
atggccattc

SEQ ID NO: 467 - DNA, 10 bp, Artificial Sequence
  cDNA sequence NM 003140.2
tggccattct

SEQ ID NO: 468 - DNA, 10 bp, Artificial Sequence
  cDNA sequence NM 003140.2
ggccattctt

SEQ ID NO: 469 - DNA, 10 bp, Artificial Sequence
  cDNA sequence NM 003140.2
gccattcttc

SEQ ID NO: 470 - DNA, 10 bp, Artificial Sequence
  cDNA sequence NM 003140.2
ccattcttcc

SEQ ID NO: 471 - DNA, 10 bp, Artificial Sequence
  cDNA sequence NM 003140.2
cattcttcca

SEQ ID NO: 472 - DNA, 10 bp, Artificial Sequence
  cDNA sequence NM 003140.2
attcttccag

SEQ ID NO: 473 - DNA, 10 bp, Artificial Sequence
  cDNA sequence NM 003140.2
ttcttccagg

SEQ ID NO: 474 - DNA, 10 bp, Artificial Sequence
  cDNA sequence NM 003140.2
tcttccagga

SEQ ID NO: 475 - DNA, 10 bp, Artificial Sequence
  cDNA sequence NM 003140.2
cttccaggag

SEQ ID NO: 476 - DNA, 10 bp, Artificial Sequence
  cDNA sequence NM 003140.2
ttccaggagg

SEQ ID NO: 477 - DNA, 10 bp, Artificial Sequence
  cDNA sequence NM 003140.2
tccaggaggc

SEQ ID NO: 478 - DNA, 10 bp, Artificial Sequence
  cDNA sequence NM 003140.2
ccaggaggca

SEQ ID NO: 479 - DNA, 10 bp, Artificial Sequence
  cDNA sequence NM 003140.2
caggaggcac

SEQ ID NO: 480 - DNA, 10 bp, Artificial Sequence
  cDNA sequence NM 003140.2
aggaggcaca

SEQ ID NO: 481 - DNA, 10 bp, Artificial Sequence
  cDNA sequence NM 003140.2
ggaggcacag

SEQ ID NO: 482 - DNA, 10 bp, Artificial Sequence
  cDNA sequence NM 003140.2
gaggcacaga

SEQ ID NO: 483 - DNA, 10 bp, Artificial Sequence
  cDNA sequence NM 003140.2
aggcacagaa

SEQ ID NO: 484 - DNA, 10 bp, Artificial Sequence
  cDNA sequence NM 003140.2
ggcacagaaa

SEQ ID NO: 485 - DNA, 10 bp, Artificial Sequence
  cDNA sequence NM 003140.2
gcacagaaat

SEQ ID NO: 486 - DNA, 10 bp, Artificial Sequence
  cDNA sequence NM 003140.2
cacagaaatt

SEQ ID NO: 487 - DNA, 10 bp, Artificial Sequence
  cDNA sequence NM 003140.2
acagaaatta

SEQ ID NO: 488 - DNA, 10 bp, Artificial Sequence
  cDNA sequence NM 003140.2
cagaaattac

SEQ ID NO: 489 - DNA, 10 bp, Artificial Sequence
  cDNA sequence NM 003140.2
agaaattaca

SEQ ID NO: 490 - DNA, 10 bp, Artificial Sequence
  cDNA sequence NM 003140.2
gaaattacag

SEQ ID NO: 491 - DNA, 10 bp, Artificial Sequence
  cDNA sequence NM 003140.2
aaattacagg

SEQ ID NO: 492 - DNA, 10 bp, Artificial Sequence
  cDNA sequence NM 003140.2
aattacaggc

SEQ ID NO: 493 - DNA, 10 bp, Artificial Sequence
  cDNA sequence NM 003140.2
attacaggcc

SEQ ID NO: 494 - DNA, 10 bp, Artificial Sequence
  cDNA sequence NM 003140.2
ttacaggcca

SEQ ID NO: 495 - DNA, 10 bp, Artificial Sequence
  cDNA sequence NM 003140.2
tacaggccat

SEQ ID NO: 496 - DNA, 10 bp, Artificial Sequence
  cDNA sequence NM 003140.2
acaggccatg

SEQ ID NO: 497 - DNA, 10 bp, Artificial Sequence
  cDNA sequence NM 003140.2
caggccatgc

SEQ ID NO: 498 - DNA, 10 bp, Artificial Sequence
  cDNA sequence NM 003140.2
aggccatgca

SEQ ID NO: 499 - DNA, 10 bp, Artificial Sequence
  cDNA sequence NM 003140.2
ggccatgcac

SEQ ID NO: 500 - DNA, 10 bp, Artificial Sequence
  cDNA sequence NM 003140.2
gccatgcaca

SEQ ID NO: 501 - DNA, 10 bp, Artificial Sequence
  cDNA sequence NM 003140.2
ccatgcacag

SEQ ID NO: 502 - DNA, 10 bp, Artificial Sequence
  cDNA sequence NM 003140.2
catgcacaga

SEQ ID NO: 503 - DNA, 10 bp, Artificial Sequence
  cDNA sequence NM 003140.2
atgcacagag

SEQ ID NO: 504 - DNA, 10 bp, Artificial Sequence
  cDNA sequence NM 003140.2
tgcacagaga

SEQ ID NO: 505 - DNA, 10 bp, Artificial Sequence
  cDNA sequence NM 003140.2
gcacagagag

SEQ ID NO: 506 - DNA, 10 bp, Artificial Sequence
  cDNA sequence NM 003140.2
cacagagaga

SEQ ID NO: 507 - DNA, 10 bp, Artificial Sequence
  cDNA sequence NM 003140.2
acagagagaa

SEQ ID NO: 508 - DNA, 10 bp, Artificial Sequence
  cDNA sequence NM 003140.2
cagagagaaa

SEQ ID NO: 509 - DNA, 10 bp, Artificial Sequence
  cDNA sequence NM 003140.2
agagagaaat

SEQ ID NO: 510 - DNA, 10 bp, Artificial Sequence
  cDNA sequence NM 003140.2
gagagaaata

SEQ ID NO: 511 - DNA, 10 bp, Artificial Sequence
  cDNA sequence NM 003140.2
agagaaatac

SEQ ID NO: 512 - DNA, 10 bp, Artificial Sequence
  cDNA sequence NM 003140.2
gagaaatacc

SEQ ID NO: 513 - DNA, 10 bp, Artificial Sequence
  cDNA sequence NM 003140.2
agaaataccc

SEQ ID NO: 514 - DNA, 10 bp, Artificial Sequence
  cDNA sequence NM 003140.2
gaaatacccg

SEQ ID NO: 515 - DNA, 10 bp, Artificial Sequence
  cDNA sequence NM 003140.2
aaatacccga

SEQ ID NO: 516 - DNA, 10 bp, Artificial Sequence
  cDNA sequence NM 003140.2
aatacccgaa

SEQ ID NO: 517 - DNA, 10 bp, Artificial Sequence
  cDNA sequence NM 003140.2
atacccgaat

SEQ ID NO: 518 - DNA, 10 bp, Artificial Sequence
  cDNA sequence NM 003140.2
tacccgaatt

SEQ ID NO: 519 - DNA, 10 bp, Artificial Sequence
  cDNA sequence NM 003140.2
acccgaatta

SEQ ID NO: 520 - DNA, 10 bp, Artificial Sequence
  cDNA sequence NM 003140.2
cccgaattat

SEQ ID NO: 521 - DNA, 10 bp, Artificial Sequence
  cDNA sequence NM 003140.2
ccgaattata

SEQ ID NO: 522 - DNA, 10 bp, Artificial Sequence
  cDNA sequence NM 003140.2
cgaattataa

SEQ ID NO: 523 - DNA, 10 bp, Artificial Sequence
  cDNA sequence NM 003140.2
gaattataag

SEQ ID NO: 524 - DNA, 10 bp, Artificial Sequence
  cDNA sequence NM 003140.2
aattataagt

SEQ ID NO: 525 - DNA, 10 bp, Artificial Sequence
  cDNA sequence NM 003140.2
attataagta

SEQ ID NO: 526 - DNA, 10 bp, Artificial Sequence
  cDNA sequence NM 003140.2
ttataagtat

SEQ ID NO: 527 - DNA, 10 bp, Artificial Sequence
  cDNA sequence NM 003140.2
tataagtatc

SEQ ID NO: 528 - DNA, 10 bp, Artificial Sequence
  cDNA sequence NM 003140.2
ataagtatcg

SEQ ID NO: 529 - DNA, 10 bp, Artificial Sequence
  cDNA sequence NM 003140.2
taagtatcga

SEQ ID NO: 530 - DNA, 10 bp, Artificial Sequence
  cDNA sequence NM 003140.2
aagtatcgac

SEQ ID NO: 531 - DNA, 10 bp, Artificial Sequence
  cDNA sequence NM 003140.2
agtatcgacc

SEQ ID NO: 532 - DNA, 10 bp, Artificial Sequence
  cDNA sequence NM 003140.2
gtatcgacct

SEQ ID NO: 533 - DNA, 10 bp, Artificial Sequence
  cDNA sequence NM 003140.2
tatcgacctc

SEQ ID NO: 534 - DNA, 10 bp, Artificial Sequence
  cDNA sequence NM 003140.2
atcgacctcg

SEQ ID NO: 535 - DNA, 10 bp, Artificial Sequence
  cDNA sequence NM 003140.2
tcgacctcgt

SEQ ID NO: 536 - DNA, 10 bp, Artificial Sequence
  cDNA sequence NM 003140.2
cgacctcgtc

SEQ ID NO: 537 - DNA, 10 bp, Artificial Sequence
  cDNA sequence NM 003140.2
gacctcgtcg

SEQ ID NO: 538 - DNA, 10 bp, Artificial Sequence
  cDNA sequence NM 003140.2
acctcgtcgg

SEQ ID NO: 539 - DNA, 10 bp, Artificial Sequence
  cDNA sequence NM 003140.2
cctcgtcgga

SEQ ID NO: 540 - DNA, 10 bp, Artificial Sequence
  cDNA sequence NM 003140.2
ctcgtcggaa

SEQ ID NO: 541 - DNA, 10 bp, Artificial Sequence
  cDNA sequence NM 003140.2
tcgtcggaag

SEQ ID NO: 542 - DNA, 10 bp, Artificial Sequence
  cDNA sequence NM 003140.2
cgtcggaagg

SEQ ID NO: 543 - DNA, 10 bp, Artificial Sequence
  cDNA sequence NM 003140.2
gtcggaaggc

SEQ ID NO: 544 - DNA, 10 bp, Artificial Sequence
  cDNA sequence NM 003140.2
tcggaaggcg

SEQ ID NO: 545 - DNA, 10 bp, Artificial Sequence
  cDNA sequence NM 003140.2
cggaaggcga

SEQ ID NO: 546 - DNA, 10 bp, Artificial Sequence
  cDNA sequence NM 003140.2
ggaaggcgaa

SEQ ID NO: 547 - DNA, 10 bp, Artificial Sequence
  cDNA sequence NM 003140.2
gaaggcgaag

SEQ ID NO: 548 - DNA, 10 bp, Artificial Sequence
  cDNA sequence NM 003140.2
aaggcgaaga

SEQ ID NO: 549 - DNA, 10 bp, Artificial Sequence
  cDNA sequence NM 003140.2
aggcgaagat

SEQ ID NO: 550 - DNA, 10 bp, Artificial Sequence
  cDNA sequence NM 003140.2
ggcgaagatg

SEQ ID NO: 551 - DNA, 10 bp, Artificial Sequence
  cDNA sequence NM 003140.2
gcgaagatgc

SEQ ID NO: 552 - DNA, 10 bp, Artificial Sequence
  cDNA sequence NM 003140.2
cgaagatgct

SEQ ID NO: 553 - DNA, 10 bp, Artificial Sequence
  cDNA sequence NM 003140.2
gaagatgctg

SEQ ID NO: 554 - DNA, 10 bp, Artificial Sequence
  cDNA sequence NM 003140.2
aagatgctgc

SEQ ID NO: 555 - DNA, 10 bp, Artificial Sequence
  cDNA sequence NM 003140.2
agatgctgcc

SEQ ID NO: 556 - DNA, 10 bp, Artificial Sequence
  cDNA sequence NM 003140.2
gatgctgccg

SEQ ID NO: 557 - DNA, 10 bp, Artificial Sequence
  cDNA sequence NM 003140.2
atgctgccga

SEQ ID NO: 558 - DNA, 10 bp, Artificial Sequence
  cDNA sequence NM 003140.2
tgctgccgaa

SEQ ID NO: 559 - DNA, 10 bp, Artificial Sequence
  cDNA sequence NM 003140.2
gctgccgaag

SEQ ID NO: 560 - DNA, 10 bp, Artificial Sequence
  cDNA sequence NM 003140.2
ctgccgaaga

SEQ ID NO: 561 - DNA, 10 bp, Artificial Sequence
  cDNA sequence NM 003140.2
tgccgaagaa

SEQ ID NO: 562 - DNA, 10 bp, Artificial Sequence
  cDNA sequence NM 003140.2
gccgaagaat

SEQ ID NO: 563 - DNA, 10 bp, Artificial Sequence
  cDNA sequence NM 003140.2
ccgaagaatt

SEQ ID NO: 564 - DNA, 10 bp, Artificial Sequence
  cDNA sequence NM 003140.2
cgaagaattg

SEQ ID NO: 565 - DNA, 10 bp, Artificial Sequence
  cDNA sequence NM 003140.2
gaagaattgc

SEQ ID NO: 566 - DNA, 10 bp, Artificial Sequence
  cDNA sequence NM 003140.2
aagaattgca

SEQ ID NO: 567 - DNA, 10 bp, Artificial Sequence
  cDNA sequence NM 003140.2
agaattgcag

SEQ ID NO: 568 - DNA, 10 bp, Artificial Sequence
  cDNA sequence NM 003140.2
gaattgcagt

SEQ ID NO: 569 - DNA, 10 bp, Artificial Sequence
  cDNA sequence NM 003140.2
aattgcagtt

SEQ ID NO: 570 - DNA, 10 bp, Artificial Sequence
  cDNA sequence NM 003140.2
attgcagttt

SEQ ID NO: 571 - DNA, 10 bp, Artificial Sequence
  cDNA sequence NM 003140.2
ttgcagtttg

SEQ ID NO: 572 - DNA, 10 bp, Artificial Sequence
  cDNA sequence NM 003140.2
tgcagtttgc

SEQ ID NO: 573 - DNA, 10 bp, Artificial Sequence
  cDNA sequence NM 003140.2
gcagtttgct

SEQ ID NO: 574 - DNA, 10 bp, Artificial Sequence
  cDNA sequence NM 003140.2
cagtttgctt

SEQ ID NO: 575 - DNA, 10 bp, Artificial Sequence
  cDNA sequence NM 003140.2
agtttgcttc

SEQ ID NO: 576 - DNA, 10 bp, Artificial Sequence
  cDNA sequence NM 003140.2
gtttgcttcc

SEQ ID NO: 577 - DNA, 10 bp, Artificial Sequence
  cDNA sequence NM 003140.2
tttgcttccc

SEQ ID NO: 578 - DNA, 10 bp, Artificial Sequence
  cDNA sequence NM 003140.2
ttgcttcccg

SEQ ID NO: 579 - DNA, 10 bp, Artificial Sequence
  cDNA sequence NM 003140.2
tgcttcccgc

SEQ ID NO: 580 - DNA, 10 bp, Artificial Sequence
  cDNA sequence NM 003140.2
gcttcccgca

SEQ ID NO: 581 - DNA, 10 bp, Artificial Sequence
  cDNA sequence NM 003140.2
cttcccgcag

SEQ ID NO: 582 - DNA, 10 bp, Artificial Sequence
  cDNA sequence NM 003140.2
ttcccgcaga

SEQ ID NO: 583 - DNA, 10 bp, Artificial Sequence
  cDNA sequence NM 003140.2
tcccgcagat

SEQ ID NO: 584 - DNA, 10 bp, Artificial Sequence
  cDNA sequence NM 003140.2
cccgcagatc

SEQ ID NO: 585 - DNA, 10 bp, Artificial Sequence
  cDNA sequence NM 003140.2
ccgcagatcc

SEQ ID NO: 586 - DNA, 10 bp, Artificial Sequence
  cDNA sequence NM 003140.2
cgcagatccc

SEQ ID NO: 587 - DNA, 10 bp, Artificial Sequence
  cDNA sequence NM 003140.2
gcagatcccg

SEQ ID NO: 588 - DNA, 10 bp, Artificial Sequence
  cDNA sequence NM 003140.2
cagatcccgc

SEQ ID NO: 589 - DNA, 10 bp, Artificial Sequence
  cDNA sequence NM 003140.2
agatcccgct

SEQ ID NO: 590 - DNA, 10 bp, Artificial Sequence
  cDNA sequence NM 003140.2
gatcccgctt

SEQ ID NO: 591 - DNA, 10 bp, Artificial Sequence
  cDNA sequence NM 003140.2
atcccgcttc

SEQ ID NO: 592 - DNA, 10 bp, Artificial Sequence
  cDNA sequence NM 003140.2
tcccgcttcg

SEQ ID NO: 593 - DNA, 10 bp, Artificial Sequence
  cDNA sequence NM 003140.2
cccgcttcgg

SEQ ID NO: 594 - DNA, 10 bp, Artificial Sequence
  cDNA sequence NM 003140.2
ccgcttcggt

SEQ ID NO: 595 - DNA, 10 bp, Artificial Sequence
  cDNA sequence NM 003140.2
cgcttcggta

SEQ ID NO: 596 - DNA, 10 bp, Artificial Sequence
  cDNA sequence NM 003140.2
gcttcggtac

SEQ ID NO: 597 - DNA, 10 bp, Artificial Sequence
  cDNA sequence NM 003140.2
cttcggtact

SEQ ID NO: 598 - DNA, 10 bp, Artificial Sequence
  cDNA sequence NM 003140.2
ttcggtactc

SEQ ID NO: 599 - DNA, 10 bp, Artificial Sequence
  cDNA sequence NM 003140.2
tcggtactct

SEQ ID NO: 600 - DNA, 10 bp, Artificial Sequence
  cDNA sequence NM 003140.2
cggtactctg

SEQ ID NO: 601 - DNA, 10 bp, Artificial Sequence
  cDNA sequence NM 003140.2
ggtactctgc

SEQ ID NO: 602 - DNA, 10 bp, Artificial Sequence
  cDNA sequence NM 003140.2
gtactctgca

SEQ ID NO: 603 - DNA, 10 bp, Artificial Sequence
  cDNA sequence NM 003140.2
tactctgcag

SEQ ID NO: 604 - DNA, 10 bp, Artificial Sequence
  cDNA sequence NM 003140.2
actctgcagc

SEQ ID NO: 605 - DNA, 10 bp, Artificial Sequence
  cDNA sequence NM 003140.2
ctctgcagcg

SEQ ID NO: 606 - DNA, 10 bp, Artificial Sequence
  cDNA sequence NM 003140.2
tctgcagcga

SEQ ID NO: 607 - DNA, 10 bp, Artificial Sequence
  cDNA sequence NM 003140.2
ctgcagcgaa

SEQ ID NO: 608 - DNA, 10 bp, Artificial Sequence
  cDNA sequence NM 003140.2
tgcagcgaag

SEQ ID NO: 609 - DNA, 10 bp, Artificial Sequence
  cDNA sequence NM 003140.2
gcagcgaagt

SEQ ID NO: 610 - DNA, 10 bp, Artificial Sequence
  cDNA sequence NM 003140.2
cagcgaagtg

SEQ ID NO: 611 - DNA, 10 bp, Artificial Sequence
  cDNA sequence NM 003140.2
agcgaagtgc

SEQ ID NO: 612 - DNA, 10 bp, Artificial Sequence
  cDNA sequence NM 003140.2
gcgaagtgca

SEQ ID NO: 613 - DNA, 10 bp, Artificial Sequence
  cDNA sequence NM 003140.2
cgaagtgcaa

SEQ ID NO: 614 - DNA, 10 bp, Artificial Sequence
  cDNA sequence NM 003140.2
gaagtgcaac

SEQ ID NO: 615 - DNA, 10 bp, Artificial Sequence
  cDNA sequence NM 003140.2
aagtgcaact

SEQ ID NO: 616 - DNA, 10 bp, Artificial Sequence
  cDNA sequence NM 003140.2
agtgcaactg

SEQ ID NO: 617 - DNA, 10 bp, Artificial Sequence
  cDNA sequence NM 003140.2
gtgcaactgg

SEQ ID NO: 618 - DNA, 10 bp, Artificial Sequence
  cDNA sequence NM 003140.2
tgcaactgga

SEQ ID NO: 619 - DNA, 10 bp, Artificial Sequence
  cDNA sequence NM 003140.2
gcaactggac

SEQ ID NO: 620 - DNA, 10 bp, Artificial Sequence
  cDNA sequence NM 003140.2
caactggaca

SEQ ID NO: 621 - DNA, 10 bp, Artificial Sequence
  cDNA sequence NM 003140.2
aactggacaa

SEQ ID NO: 622 - DNA, 10 bp, Artificial Sequence
  cDNA sequence NM 003140.2
actggacaac

SEQ ID NO: 623 - DNA, 10 bp, Artificial Sequence
  cDNA sequence NM 003140.2
ctggacaaca

SEQ ID NO: 624 - DNA, 10 bp, Artificial Sequence
  cDNA sequence NM 003140.2
tggacaacag

SEQ ID NO: 625 - DNA, 10 bp, Artificial Sequence
  cDNA sequence NM 003140.2
ggacaacagg

SEQ ID NO: 626 - DNA, 10 bp, Artificial Sequence
  cDNA sequence NM 003140.2
gacaacaggt

SEQ ID NO: 627 - DNA, 10 bp, Artificial Sequence
  cDNA sequence NM 003140.2
acaacaggtt

SEQ ID NO: 628 - DNA, 10 bp, Artificial Sequence
  cDNA sequence NM 003140.2
caacaggttg

SEQ ID NO: 629 - DNA, 10 bp, Artificial Sequence
  cDNA sequence NM 003140.2
aacaggttgt

SEQ ID NO: 630 - DNA, 10 bp, Artificial Sequence
  cDNA sequence NM 003140.2
acaggttgta

SEQ ID NO: 631 - DNA, 10 bp, Artificial Sequence
  cDNA sequence NM 003140.2
caggttgtac

SEQ ID NO: 632 - DNA, 10 bp, Artificial Sequence
  cDNA sequence NM 003140.2
aggttgtaca

SEQ ID NO: 633 - DNA, 10 bp, Artificial Sequence
  cDNA sequence NM 003140.2
ggttgtacag

SEQ ID NO: 634 - DNA, 10 bp, Artificial Sequence
  cDNA sequence NM 003140.2
gttgtacagg

SEQ ID NO: 635 - DNA, 10 bp, Artificial Sequence
  cDNA sequence NM 003140.2
ttgtacaggg

SEQ ID NO: 636 - DNA, 10 bp, Artificial Sequence
  cDNA sequence NM 003140.2
tgtacaggga

SEQ ID NO: 637 - DNA, 10 bp, Artificial Sequence
  cDNA sequence NM 003140.2
gtacagggat

SEQ ID NO: 638 - DNA, 10 bp, Artificial Sequence
  cDNA sequence NM 003140.2
tacagggatg

SEQ ID NO: 639 - DNA, 10 bp, Artificial Sequence
  cDNA sequence NM 003140.2
acagggatga

SEQ ID NO: 640 - DNA, 10 bp, Artificial Sequence
  cDNA sequence NM 003140.2
cagggatgac

SEQ ID NO: 641 - DNA, 10 bp, Artificial Sequence
  cDNA sequence NM 003140.2
agggatgact

SEQ ID NO: 642 - DNA, 10 bp, Artificial Sequence
  cDNA sequence NM 003140.2
gggatgactg

SEQ ID NO: 643 - DNA, 10 bp, Artificial Sequence
  cDNA sequence NM 003140.2
ggatgactgt

SEQ ID NO: 644 - DNA, 10 bp, Artificial Sequence
  cDNA sequence NM 003140.2
gatgactgta

SEQ ID NO: 645 - DNA, 10 bp, Artificial Sequence
  cDNA sequence NM 003140.2
atgactgtac

SEQ ID NO: 646 - DNA, 10 bp, Artificial Sequence
  cDNA sequence NM 003140.2
tgactgtacg

SEQ ID NO: 647 - DNA, 10 bp, Artificial Sequence
  cDNA sequence NM 003140.2
gactgtacga

SEQ ID NO: 648 - DNA, 10 bp, Artificial Sequence
  cDNA sequence NM 003140.2
actgtacgaa

SEQ ID NO: 649 - DNA, 10 bp, Artificial Sequence
  cDNA sequence NM 003140.2
ctgtacgaaa

SEQ ID NO: 650 - DNA, 10 bp, Artificial Sequence
  cDNA sequence NM 003140.2
tgtacgaaag

SEQ ID NO: 651 - DNA, 10 bp, Artificial Sequence
  cDNA sequence NM 003140.2
gtacgaaagc

SEQ ID NO: 652 - DNA, 10 bp, Artificial Sequence
  cDNA sequence NM 003140.2
tacgaaagcc

SEQ ID NO: 653 - DNA, 10 bp, Artificial Sequence
  cDNA sequence NM 003140.2
acgaaagcca

SEQ ID NO: 654 - DNA, 10 bp, Artificial Sequence
  cDNA sequence NM 003140.2
cgaaagccac

SEQ ID NO: 655 - DNA, 10 bp, Artificial Sequence
  cDNA sequence NM 003140.2
gaaagccaca

SEQ ID NO: 656 - DNA, 10 bp, Artificial Sequence
  cDNA sequence NM 003140.2
aaagccacac

SEQ ID NO: 657 - DNA, 10 bp, Artificial Sequence
  cDNA sequence NM 003140.2
aagccacaca

SEQ ID NO: 658 - DNA, 10 bp, Artificial Sequence
  cDNA sequence NM 003140.2
agccacacac

SEQ ID NO: 659 - DNA, 10 bp, Artificial Sequence
  cDNA sequence NM 003140.2
gccacacact

SEQ ID NO: 660 - DNA, 10 bp, Artificial Sequence
  cDNA sequence NM 003140.2
ccacacactc

SEQ ID NO: 661 - DNA, 10 bp, Artificial Sequence
  cDNA sequence NM 003140.2
cacacactca

SEQ ID NO: 662 - DNA, 10 bp, Artificial Sequence
  cDNA sequence NM 003140.2
acacactcaa

SEQ ID NO: 663 - DNA, 10 bp, Artificial Sequence
  cDNA sequence NM 003140.2
cacactcaag

SEQ ID NO: 664 - DNA, 10 bp, Artificial Sequence
  cDNA sequence NM 003140.2
acactcaaga

SEQ ID NO: 665 - DNA, 10 bp, Artificial Sequence
  cDNA sequence NM 003140.2
cactcaagaa

SEQ ID NO: 666 - DNA, 10 bp, Artificial Sequence
  cDNA sequence NM 003140.2
actcaagaat

SEQ ID NO: 667 - DNA, 10 bp, Artificial Sequence
  cDNA sequence NM 003140.2
ctcaagaatg

SEQ ID NO: 668 - DNA, 10 bp, Artificial Sequence
  cDNA sequence NM 003140.2
tcaagaatgg

SEQ ID NO: 669 - DNA, 10 bp, Artificial Sequence
  cDNA sequence NM 003140.2
caagaatgga

SEQ ID NO: 670 - DNA, 10 bp, Artificial Sequence
  cDNA sequence NM 003140.2
aagaatggag

SEQ ID NO: 671 - DNA, 10 bp, Artificial Sequence
  cDNA sequence NM 003140.2
agaatggagc

SEQ ID NO: 672 - DNA, 10 bp, Artificial Sequence
  cDNA sequence NM 003140.2
gaatggagca

SEQ ID NO: 673 - DNA, 10 bp, Artificial Sequence
  cDNA sequence NM 003140.2
aatggagcac

SEQ ID NO: 674 - DNA, 10 bp, Artificial Sequence
  cDNA sequence NM 003140.2
atggagcacc

SEQ ID NO: 675 - DNA, 10 bp, Artificial Sequence
  cDNA sequence NM 003140.2
tggagcacca

SEQ ID NO: 676 - DNA, 10 bp, Artificial Sequence
  cDNA sequence NM 003140.2
ggagcaccag

SEQ ID NO: 677 - DNA, 10 bp, Artificial Sequence
  cDNA sequence NM 003140.2
gagcaccagc

SEQ ID NO: 678 - DNA, 10 bp, Artificial Sequence
  cDNA sequence NM 003140.2
agcaccagct

SEQ ID NO: 679 - DNA, 10 bp, Artificial Sequence
  cDNA sequence NM 003140.2
gcaccagcta

SEQ ID NO: 680 - DNA, 10 bp, Artificial Sequence
  cDNA sequence NM 003140.2
caccagctag

SEQ ID NO: 681 - DNA, 10 bp, Artificial Sequence
  cDNA sequence NM 003140.2
accagctagg

SEQ ID NO: 682 - DNA, 10 bp, Artificial Sequence
  cDNA sequence NM 003140.2
ccagctaggc

SEQ ID NO: 683 - DNA, 10 bp, Artificial Sequence
  cDNA sequence NM 003140.2
cagctaggcc

SEQ ID NO: 684 - DNA, 10 bp, Artificial Sequence
  cDNA sequence NM 003140.2
agctaggcca

SEQ ID NO: 685 - DNA, 10 bp, Artificial Sequence
  cDNA sequence NM 003140.2
gctaggccac

SEQ ID NO: 686 - DNA, 10 bp, Artificial Sequence
  cDNA sequence NM 003140.2
ctaggccact

SEQ ID NO: 687 - DNA, 10 bp, Artificial Sequence
  cDNA sequence NM 003140.2
taggccactt

SEQ ID NO: 688 - DNA, 10 bp, Artificial Sequence
  cDNA sequence NM 003140.2
aggccactta

SEQ ID NO: 689 - DNA, 10 bp, Artificial Sequence
  cDNA sequence NM 003140.2
ggccacttac

SEQ ID NO: 690 - DNA, 10 bp, Artificial Sequence
  cDNA sequence NM 003140.2
gccacttacc

SEQ ID NO: 691 - DNA, 10 bp, Artificial Sequence
  cDNA sequence NM 003140.2
ccacttaccg

SEQ ID NO: 692 - DNA, 10 bp, Artificial Sequence
  cDNA sequence NM 003140.2
cacttaccgc

SEQ ID NO: 693 - DNA, 10 bp, Artificial Sequence
  cDNA sequence NM 003140.2
acttaccgcc

SEQ ID NO: 694 - DNA, 10 bp, Artificial Sequence
  cDNA sequence NM 003140.2
cttaccgccc

SEQ ID NO: 695 - DNA, 10 bp, Artificial Sequence
  cDNA sequence NM 003140.2
ttaccgccca

SEQ ID NO: 696 - DNA, 10 bp, Artificial Sequence
  cDNA sequence NM 003140.2
taccgcccat

SEQ ID NO: 697 - DNA, 10 bp, Artificial Sequence
  cDNA sequence NM 003140.2
accgcccatc

SEQ ID NO: 698 - DNA, 10 bp, Artificial Sequence
  cDNA sequence NM 003140.2
ccgcccatca

SEQ ID NO: 699 - DNA, 10 bp, Artificial Sequence
  cDNA sequence NM 003140.2
cgcccatcaa

SEQ ID NO: 700 - DNA, 10 bp, Artificial Sequence
  cDNA sequence NM 003140.2
gcccatcaac

SEQ ID NO: 701 - DNA, 10 bp, Artificial Sequence
  cDNA sequence NM 003140.2
cccatcaacg

SEQ ID NO: 702 - DNA, 10 bp, Artificial Sequence
  cDNA sequence NM 003140.2
ccatcaacgc

SEQ ID NO: 703 - DNA, 10 bp, Artificial Sequence
  cDNA sequence NM 003140.2
catcaacgca

SEQ ID NO: 704 - DNA, 10 bp, Artificial Sequence
  cDNA sequence NM 003140.2
atcaacgcag

SEQ ID NO: 705 - DNA, 10 bp, Artificial Sequence
  cDNA sequence NM 003140.2
tcaacgcagc

SEQ ID NO: 706 - DNA, 10 bp, Artificial Sequence
  cDNA sequence NM 003140.2
caacgcagcc

SEQ ID NO: 707 - DNA, 10 bp, Artificial Sequence
  cDNA sequence NM 003140.2
cgcagccagc

SEQ ID NO: 708 - DNA, 10 bp, Artificial Sequence
  cDNA sequence NM 003140.2
gcagccagct

SEQ ID NO: 709 - DNA, 10 bp, Artificial Sequence
  cDNA sequence NM 003140.2
cagccagctc

SEQ ID NO: 710 - DNA, 10 bp, Artificial Sequence
  cDNA sequence NM 003140.2
agccagctca

SEQ ID NO: 711 - DNA, 10 bp, Artificial Sequence
  cDNA sequence NM 003140.2
gccagctcac

SEQ ID NO: 712 - DNA, 10 bp, Artificial Sequence
  cDNA sequence NM 003140.2
ccagctcacc

SEQ ID NO: 713 - DNA, 10 bp, Artificial Sequence
  cDNA sequence NM 003140.2
cagctcaccg

SEQ ID NO: 714 - DNA, 10 bp, Artificial Sequence
  cDNA sequence NM 003140.2
agctcaccgc

SEQ ID NO: 715 - DNA, 10 bp, Artificial Sequence
  cDNA sequence NM 003140.2
gctcaccgca

SEQ ID NO: 716 - DNA, 10 bp, Artificial Sequence
  cDNA sequence NM 003140.2
ctcaccgcag

SEQ ID NO: 717 - DNA, 10 bp, Artificial Sequence
  cDNA sequence NM 003140.2
tcaccgcagc

SEQ ID NO: 718 - DNA, 10 bp, Artificial Sequence
  cDNA sequence NM 003140.2
caccgcagca

SEQ ID NO: 719 - DNA, 10 bp, Artificial Sequence
  cDNA sequence NM 003140.2
accgcagcaa

SEQ ID NO: 720 - DNA, 10 bp, Artificial Sequence
  cDNA sequence NM 003140.2
ccgcagcaac

SEQ ID NO: 721 - DNA, 10 bp, Artificial Sequence
  cDNA sequence NM 003140.2
cgcagcaacg

SEQ ID NO: 722 - DNA, 10 bp, Artificial Sequence
  cDNA sequence NM 003140.2
gcagcaacgg

SEQ ID NO: 723 - DNA, 10 bp, Artificial Sequence
  cDNA sequence NM 003140.2
cagcaacggg

SEQ ID NO: 724 - DNA, 10 bp, Artificial Sequence
  cDNA sequence NM 003140.2
agcaacggga

SEQ ID NO: 725 - DNA, 10 bp, Artificial Sequence
  cDNA sequence NM 003140.2
gcaacgggac

SEQ ID NO: 726 - DNA, 10 bp, Artificial Sequence
  cDNA sequence NM 003140.2
caacgggacc

SEQ ID NO: 727 - DNA, 10 bp, Artificial Sequence
  cDNA sequence NM 003140.2
aacgggaccg

SEQ ID NO: 728 - DNA, 10 bp, Artificial Sequence
  cDNA sequence NM 003140.2
acgggaccgc

SEQ ID NO: 729 - DNA, 10 bp, Artificial Sequence
  cDNA sequence NM 003140.2
cgggaccgct

SEQ ID NO: 730 - DNA, 10 bp, Artificial Sequence
  cDNA sequence NM 003140.2
gggaccgcta

SEQ ID NO: 731 - DNA, 10 bp, Artificial Sequence
  cDNA sequence NM 003140.2
ggaccgctac

SEQ ID NO: 732 - DNA, 10 bp, Artificial Sequence
  cDNA sequence NM 003140.2
gaccgctaca

SEQ ID NO: 733 - DNA, 10 bp, Artificial Sequence
  cDNA sequence NM 003140.2
accgctacag

SEQ ID NO: 734 - DNA, 10 bp, Artificial Sequence
  cDNA sequence NM 003140.2
ccgctacagc

SEQ ID NO: 735 - DNA, 10 bp, Artificial Sequence
  cDNA sequence NM 003140.2
cgctacagcc

SEQ ID NO: 736 - DNA, 10 bp, Artificial Sequence
  cDNA sequence NM 003140.2
gctacagcca

SEQ ID NO: 737 - DNA, 10 bp, Artificial Sequence
  cDNA sequence NM 003140.2
ctacagccac

SEQ ID NO: 738 - DNA, 10 bp, Artificial Sequence
  cDNA sequence NM 003140.2
tacagccact

SEQ ID NO: 739 - DNA, 10 bp, Artificial Sequence
  cDNA sequence NM 003140.2
acagccactg

SEQ ID NO: 740 - DNA, 10 bp, Artificial Sequence
  cDNA sequence NM 003140.2
cagccactgg

SEQ ID NO: 741 - DNA, 10 bp, Artificial Sequence
  cDNA sequence NM 003140.2
agccactgga

SEQ ID NO: 742 - DNA, 10 bp, Artificial Sequence
  cDNA sequence NM 003140.2
gccactggac

SEQ ID NO: 743 - DNA, 10 bp, Artificial Sequence
  cDNA sequence NM 003140.2
ccactggaca

SEQ ID NO: 744 - DNA, 10 bp, Artificial Sequence
  cDNA sequence NM 003140.2
cactggacaa

SEQ ID NO: 745 - DNA, 10 bp, Artificial Sequence
  cDNA sequence NM 003140.2
actggacaaa

SEQ ID NO: 746 - DNA, 10 bp, Artificial Sequence
  cDNA sequence NM 003140.2
ctggacaaag

SEQ ID NO: 747 - DNA, 10 bp, Artificial Sequence
  cDNA sequence NM 003140.2
tggacaaagc

SEQ ID NO: 748 - DNA, 10 bp, Artificial Sequence
  cDNA sequence NM 003140.2
ggacaaagct

SEQ ID NO: 749 - DNA, 10 bp, Artificial Sequence
  cDNA sequence NM 003140.2
gacaaagctg

SEQ ID NO: 750 - DNA, 10 bp, Artificial Sequence
  cDNA sequence NM 003140.2
acaaagctgt

SEQ ID NO: 751 - DNA, 10 bp, Artificial Sequence
  cDNA sequence NM 003140.2
caaagctgta

SEQ ID NO: 752 - DNA, 10 bp, Artificial Sequence
  cDNA sequence NM 003140.2
aaagctgtag

SEQ ID NO: 753 - DNA, 10 bp, Artificial Sequence
  cDNA sequence NM 003140.2
aagctgtagg

SEQ ID NO: 754 - DNA, 10 bp, Artificial Sequence
  cDNA sequence NM 003140.2
agctgtagga

SEQ ID NO: 755 - DNA, 10 bp, Artificial Sequence
  cDNA sequence NM 003140.2
gctgtaggac

SEQ ID NO: 756 - DNA, 10 bp, Artificial Sequence
  cDNA sequence NM 003140.2
ctgtaggaca

SEQ ID NO: 757 - DNA, 10 bp, Artificial Sequence
  cDNA sequence NM 003140.2
tgtaggacaa

SEQ ID NO: 758 - DNA, 10 bp, Artificial Sequence
  cDNA sequence NM 003140.2
gtaggacaat

SEQ ID NO: 759 - DNA, 10 bp, Artificial Sequence
  cDNA sequence NM 003140.2
taggacaatc

SEQ ID NO: 760 - DNA, 10 bp, Artificial Sequence
  cDNA sequence NM 003140.2
aggacaatcg

SEQ ID NO: 761 - DNA, 10 bp, Artificial Sequence
  cDNA sequence NM 003140.2
ggacaatcgg

SEQ ID NO: 762 - DNA, 10 bp, Artificial Sequence
  cDNA sequence NM 003140.2
gacaatcggg

SEQ ID NO: 763 - DNA, 10 bp, Artificial Sequence
  cDNA sequence NM 003140.2
acaatcgggt

SEQ ID NO: 764 - DNA, 10 bp, Artificial Sequence
  cDNA sequence NM 003140.2
caatcgggta

SEQ ID NO: 765 - DNA, 10 bp, Artificial Sequence
  cDNA sequence NM 003140.2
aatcgggtaa

SEQ ID NO: 766 - DNA, 10 bp, Artificial Sequence
  cDNA sequence NM 003140.2
atcgggtaac

SEQ ID NO: 767 - DNA, 10 bp, Artificial Sequence
  cDNA sequence NM 003140.2
tcgggtaaca

SEQ ID NO: 768 - DNA, 10 bp, Artificial Sequence
  cDNA sequence NM 003140.2
cgggtaacat

SEQ ID NO: 769 - DNA, 10 bp, Artificial Sequence
  cDNA sequence NM 003140.2
gggtaacatt

SEQ ID NO: 770 - DNA, 10 bp, Artificial Sequence
  cDNA sequence NM 003140.2
ggtaacattg

SEQ ID NO: 771 - DNA, 10 bp, Artificial Sequence
  cDNA sequence NM 003140.2
gtaacattgg

SEQ ID NO: 772 - DNA, 10 bp, Artificial Sequence
  cDNA sequence NM 003140.2
taacattggc

SEQ ID NO: 773 - DNA, 10 bp, Artificial Sequence
  cDNA sequence NM 003140.2
aacattggct

SEQ ID NO: 774 - DNA, 10 bp, Artificial Sequence
  cDNA sequence NM 003140.2
acattggcta

SEQ ID NO: 775 - DNA, 10 bp, Artificial Sequence
  cDNA sequence NM 003140.2
cattggctac

SEQ ID NO: 776 - DNA, 10 bp, Artificial Sequence
  cDNA sequence NM 003140.2
attggctaca

SEQ ID NO: 777 - DNA, 10 bp, Artificial Sequence
  cDNA sequence NM 003140.2
ttggctacaa

SEQ ID NO: 778 - DNA, 10 bp, Artificial Sequence
  cDNA sequence NM 003140.2
tggctacaaa

SEQ ID NO: 779 - DNA, 10 bp, Artificial Sequence
  cDNA sequence NM 003140.2
ggctacaaag

SEQ ID NO: 780 - DNA, 10 bp, Artificial Sequence
  cDNA sequence NM 003140.2
gctacaaaga

SEQ ID NO: 781 - DNA, 10 bp, Artificial Sequence
  cDNA sequence NM 003140.2
ctacaaagac

SEQ ID NO: 782 - DNA, 10 bp, Artificial Sequence
  cDNA sequence NM 003140.2
tacaaagacc

SEQ ID NO: 783 - DNA, 10 bp, Artificial Sequence
  cDNA sequence NM 003140.2
acaaagacct

SEQ ID NO: 784 - DNA, 10 bp, Artificial Sequence
  cDNA sequence NM 003140.2
caaagaccta

SEQ ID NO: 785 - DNA, 10 bp, Artificial Sequence
  cDNA sequence NM 003140.2
aaagacctac

SEQ ID NO: 786 - DNA, 10 bp, Artificial Sequence
  cDNA sequence NM 003140.2
aagacctacc

SEQ ID NO: 787 - DNA, 10 bp, Artificial Sequence
  cDNA sequence NM 003140.2
agacctacct

SEQ ID NO: 788 - DNA, 10 bp, Artificial Sequence
  cDNA sequence NM 003140.2
gacctaccta

SEQ ID NO: 789 - DNA, 10 bp, Artificial Sequence
  cDNA sequence NM 003140.2
acctacctag

SEQ ID NO: 790 - DNA, 10 bp, Artificial Sequence
  cDNA sequence NM 003140.2
cctacctaga

SEQ ID NO: 791 - DNA, 10 bp, Artificial Sequence
  cDNA sequence NM 003140.2
ctacctagat

SEQ ID NO: 792 - DNA, 10 bp, Artificial Sequence
  cDNA sequence NM 003140.2
tacctagatg

SEQ ID NO: 793 - DNA, 10 bp, Artificial Sequence
  cDNA sequence NM 003140.2
acctagatgc

SEQ ID NO: 794 - DNA, 10 bp, Artificial Sequence
  cDNA sequence NM 003140.2
cctagatgct

SEQ ID NO: 795 - DNA, 10 bp, Artificial Sequence
  cDNA sequence NM 003140.2
ctagatgctc

SEQ ID NO: 796 - DNA, 10 bp, Artificial Sequence
  cDNA sequence NM 003140.2
tagatgctcc

SEQ ID NO: 797 - DNA, 10 bp, Artificial Sequence
  cDNA sequence NM 003140.2
agatgctcct

SEQ ID NO: 798 - DNA, 10 bp, Artificial Sequence
  cDNA sequence NM 003140.2
gatgctcctt

SEQ ID NO: 799 - DNA, 10 bp, Artificial Sequence
  cDNA sequence NM 003140.2
atgctccttt

SEQ ID NO: 800 - DNA, 10 bp, Artificial Sequence
  cDNA sequence NM 003140.2
tgctcctttt

SEQ ID NO: 801 - DNA, 10 bp, Artificial Sequence
  cDNA sequence NM 003140.2
gctccttttt

SEQ ID NO: 802 - DNA, 10 bp, Artificial Sequence
  cDNA sequence NM 003140.2
ctccttttta

SEQ ID NO: 803 - DNA, 10 bp, Artificial Sequence
  cDNA sequence NM 003140.2
tcctttttac

SEQ ID NO: 804 - DNA, 10 bp, Artificial Sequence
  cDNA sequence NM 003140.2
cctttttacg

SEQ ID NO: 805 - DNA, 10 bp, Artificial Sequence
  cDNA sequence NM 003140.2
ctttttacga

SEQ ID NO: 806 - DNA, 10 bp, Artificial Sequence
  cDNA sequence NM 003140.2
tttttacgat

SEQ ID NO: 807 - DNA, 10 bp, Artificial Sequence
  cDNA sequence NM 003140.2
ttttacgata

SEQ ID NO: 808 - DNA, 10 bp, Artificial Sequence
  cDNA sequence NM 003140.2
tttacgataa

SEQ ID NO: 809 - DNA, 10 bp, Artificial Sequence
  cDNA sequence NM 003140.2
ttacgataac

SEQ ID NO: 810 - DNA, 10 bp, Artificial Sequence
  cDNA sequence NM 003140.2
tacgataact

SEQ ID NO: 811 - DNA, 10 bp, Artificial Sequence
  cDNA sequence NM 003140.2
acgataactt

SEQ ID NO: 812 - DNA, 10 bp, Artificial Sequence
  cDNA sequence NM 003140.2
cgataactta

SEQ ID NO: 813 - DNA, 10 bp, Artificial Sequence
  cDNA sequence NM 003140.2
gataacttac

SEQ ID NO: 814 - DNA, 10 bp, Artificial Sequence
  cDNA sequence NM 003140.2
ataacttaca

SEQ ID NO: 815 - DNA, 10 bp, Artificial Sequence
  cDNA sequence NM 003140.2
taacttacag

SEQ ID NO: 816 - DNA, 10 bp, Artificial Sequence
  cDNA sequence NM 003140.2
aacttacagc

SEQ ID NO: 817 - DNA, 10 bp, Artificial Sequence
  cDNA sequence NM 003140.2
acttacagcc

SEQ ID NO: 818 - DNA, 10 bp, Artificial Sequence
  cDNA sequence NM 003140.2
cttacagccc

SEQ ID NO: 819 - DNA, 10 bp, Artificial Sequence
  cDNA sequence NM 003140.2
ttacagccct

SEQ ID NO: 820 - DNA, 10 bp, Artificial Sequence
  cDNA sequence NM 003140.2
tacagccctc

SEQ ID NO: 821 - DNA, 10 bp, Artificial Sequence
  cDNA sequence NM 003140.2
acagccctca

SEQ ID NO: 822 - DNA, 10 bp, Artificial Sequence
  cDNA sequence NM 003140.2
cagccctcac

SEQ ID NO: 823 - DNA, 10 bp, Artificial Sequence
  cDNA sequence NM 003140.2
agccctcact

SEQ ID NO: 824 - DNA, 10 bp, Artificial Sequence
  cDNA sequence NM 003140.2
gccctcactt

SEQ ID NO: 825 - DNA, 10 bp, Artificial Sequence
  cDNA sequence NM 003140.2
ccctcacttt

SEQ ID NO: 826 - DNA, 10 bp, Artificial Sequence
  cDNA sequence NM 003140.2
cctcactttc

SEQ ID NO: 827 - DNA, 10 bp, Artificial Sequence
  cDNA sequence NM 003140.2
ctcactttct

SEQ ID NO: 828 - DNA, 10 bp, Artificial Sequence
  cDNA sequence NM 003140.2
tcactttctt

SEQ ID NO: 829 - DNA, 10 bp, Artificial Sequence
  cDNA sequence NM 003140.2
cactttctta

SEQ ID NO: 830 - DNA, 10 bp, Artificial Sequence
  cDNA sequence NM 003140.2
actttcttat

SEQ ID NO: 831 - DNA, 10 bp, Artificial Sequence
  cDNA sequence NM 003140.2
ctttcttatg

SEQ ID NO: 832 - DNA, 10 bp, Artificial Sequence
  cDNA sequence NM 003140.2
tttcttatgt

SEQ ID NO: 833 - DNA, 10 bp, Artificial Sequence
  cDNA sequence NM 003140.2
ttcttatgtt

SEQ ID NO: 834 - DNA, 10 bp, Artificial Sequence
  cDNA sequence NM 003140.2
tcttatgttt

SEQ ID NO: 835 - DNA, 10 bp, Artificial Sequence
  cDNA sequence NM 003140.2
cttatgttta

SEQ ID NO: 836 - DNA, 10 bp, Artificial Sequence
  cDNA sequence NM 003140.2
ttatgtttag

SEQ ID NO: 837 - DNA, 10 bp, Artificial Sequence
  cDNA sequence NM 003140.2
tatgtttagt

SEQ ID NO: 838 - DNA, 10 bp, Artificial Sequence
  cDNA sequence NM 003140.2
atgtttagtt

SEQ ID NO: 839 - DNA, 10 bp, Artificial Sequence
  cDNA sequence NM 003140.2
tgtttagttt

SEQ ID NO: 840 - DNA, 10 bp, Artificial Sequence
  cDNA sequence NM 003140.2
gtttagtttc

SEQ ID NO: 841 - DNA, 10 bp, Artificial Sequence
  cDNA sequence NM 003140.2
tttagtttca

SEQ ID NO: 842 - DNA, 10 bp, Artificial Sequence
  cDNA sequence NM 003140.2
ttagtttcaa

SEQ ID NO: 843 - DNA, 10 bp, Artificial Sequence
  cDNA sequence NM 003140.2
tagtttcaat

SEQ ID NO: 844 - DNA, 10 bp, Artificial Sequence
  cDNA sequence NM 003140.2
agtttcaata

SEQ ID NO: 845 - DNA, 10 bp, Artificial Sequence
  cDNA sequence NM 003140.2
gtttcaatat

SEQ ID NO: 846 - DNA, 10 bp, Artificial Sequence
  cDNA sequence NM 003140.2
tttcaatatt

SEQ ID NO: 847 - DNA, 10 bp, Artificial Sequence
  cDNA sequence NM 003140.2
ttcaatattg

SEQ ID NO: 848 - DNA, 10 bp, Artificial Sequence
  cDNA sequence NM 003140.2
tcaatattgt

SEQ ID NO: 849 - DNA, 10 bp, Artificial Sequence
  cDNA sequence NM 003140.2
caatattgtt

SEQ ID NO: 850 - DNA, 10 bp, Artificial Sequence
  cDNA sequence NM 003140.2
aatattgttt

SEQ ID NO: 851 - DNA, 10 bp, Artificial Sequence
  cDNA sequence NM 003140.2
atattgtttt

SEQ ID NO: 852 - DNA, 10 bp, Artificial Sequence
  cDNA sequence NM 003140.2
tattgttttc

SEQ ID NO: 853 - DNA, 10 bp, Artificial Sequence
  cDNA sequence NM 003140.2
attgttttct

SEQ ID NO: 854 - DNA, 10 bp, Artificial Sequence
  cDNA sequence NM 003140.2
ttgttttctt

SEQ ID NO: 855 - DNA, 10 bp, Artificial Sequence
  cDNA sequence NM 003140.2
tgttttcttt

SEQ ID NO: 856 - DNA, 10 bp, Artificial Sequence
  cDNA sequence NM 003140.2
gttttctttt

SEQ ID NO: 857 - DNA, 10 bp, Artificial Sequence
  cDNA sequence NM 003140.2
ttttcttttc

SEQ ID NO: 858 - DNA, 10 bp, Artificial Sequence
  cDNA sequence NM 003140.2
tttcttttct

SEQ ID NO: 859 - DNA, 10 bp, Artificial Sequence
  cDNA sequence NM 003140.2
ttcttttctc

SEQ ID NO: 860 - DNA, 10 bp, Artificial Sequence
  cDNA sequence NM 003140.2
tcttttctct

SEQ ID NO: 861 - DNA, 10 bp, Artificial Sequence
  cDNA sequence NM 003140.2
cttttctctg

SEQ ID NO: 862 - DNA, 10 bp, Artificial Sequence
  cDNA sequence NM 003140.2
ttttctctgg

SEQ ID NO: 863 - DNA, 10 bp, Artificial Sequence
  cDNA sequence NM 003140.2
tttctctggc

SEQ ID NO: 864 - DNA, 10 bp, Artificial Sequence
  cDNA sequence NM 003140.2
ttctctggct

SEQ ID NO: 865 - DNA, 10 bp, Artificial Sequence
  cDNA sequence NM 003140.2
tctctggcta

SEQ ID NO: 866 - DNA, 10 bp, Artificial Sequence
  cDNA sequence NM 003140.2
ctctggctaa

SEQ ID NO: 867 - DNA, 10 bp, Artificial Sequence
  cDNA sequence NM 003140.2
tctggctaat

SEQ ID NO: 868 - DNA, 10 bp, Artificial Sequence
  cDNA sequence NM 003140.2
ctggctaata

SEQ ID NO: 869 - DNA, 10 bp, Artificial Sequence
  cDNA sequence NM 003140.2
tggctaataa

SEQ ID NO: 870 - DNA, 10 bp, Artificial Sequence
  cDNA sequence NM 003140.2
ggctaataaa

SEQ ID NO: 871 - DNA, 10 bp, Artificial Sequence
  cDNA sequence NM 003140.2
gctaataaag

SEQ ID NO: 872 - DNA, 10 bp, Artificial Sequence
  cDNA sequence NM 003140.2
ctaataaagg

SEQ ID NO: 873 - DNA, 10 bp, Artificial Sequence
  cDNA sequence NM 003140.2
taataaaggc

SEQ ID NO: 874 - DNA, 10 bp, Artificial Sequence
  cDNA sequence NM 003140.2
aataaaggcc

SEQ ID NO: 875 - DNA, 10 bp, Artificial Sequence
  cDNA sequence NM 003140.2
ataaaggcct

SEQ ID NO: 876 - DNA, 10 bp, Artificial Sequence
  cDNA sequence NM 003140.2
taaaggcctt

SEQ ID NO: 877 - DNA, 10 bp, Artificial Sequence
  cDNA sequence NM 003140.2
aaaggcctta

SEQ ID NO: 878 - DNA, 10 bp, Artificial Sequence
  cDNA sequence NM 003140.2
aaggccttat

SEQ ID NO: 879 - DNA, 10 bp, Artificial Sequence
  cDNA sequence NM 003140.2
aggccttatt

SEQ ID NO: 880 - DNA, 10 bp, Artificial Sequence
  cDNA sequence NM 003140.2
ggccttattc

SEQ ID NO: 881 - DNA, 10 bp, Artificial Sequence
  cDNA sequence NM 003140.2
gccttattca

SEQ ID NO: 882 - DNA, 10 bp, Artificial Sequence
  cDNA sequence NM 003140.2
ccttattcat

SEQ ID NO: 883 - DNA, 10 bp, Artificial Sequence
  cDNA sequence NM 003140.2
cttattcatt

SEQ ID NO: 884 - DNA, 10 bp, Artificial Sequence
  cDNA sequence NM 003140.2
ttattcattt

SEQ ID NO: 885 - DNA, 10 bp, Artificial Sequence
  cDNA sequence NM 003140.2
tattcatttc

SEQ ID NO: 886 - DNA, 10 bp, Artificial Sequence
  cDNA sequence NM 003140.2
attcatttca

SEQ ID NO: 887 - DNA, 20 bp, Artificial Sequence
  Target Site A
caatgcaatcatatgcttct

SEQ ID NO: 888 - DNA, 20 bp, Artificial Sequence
  ASO-A
guuacgttagtatacgaaga

SEQ ID NO: 889 - DNA, 20 bp, Artificial Sequence
  Target Site B
gaaaacagtaaaggcaacgt

SEQ ID NO: 890 - DNA, 20 bp, Artificial Sequence
  ASO-B
cuuuugtcatttccguugca

SEQ ID NO: 891 - DNA, 20 bp, Artificial Sequence
  Target Site C
acccatgaacgcattcatcg

SEQ ID NO: 892 - DNA, 20 bp, Artificial Sequence
  ASO-C
uggguacttgcgtaagtagc

SEQ ID NO: 893 - DNA, 20 bp, Artificial Sequence
  Target Site D
aagccacacactcaagaatg

SEQ ID NO: 894 - DNA, 20 bp, Artificial Sequence
  ASO-D
uucggtgtgtgagttcuuac

SEQ ID NO: 895 - DNA, 20 bp, Artificial Sequence
  Target Site E
taaaggccttattcatttca

SEQ ID NO: 896 - DNA, 20 bp, Artificial Sequence
  ASO-E
auuuccggaataagtaaagt

SEQ ID NO: 897 - DNA, 21 bp, Artificial Sequence
  Antisense Therapy ODNs +1 to +21
agcagaagcatatgattgcat

SEQ ID NO: 898 - DNA, 23 bp, Artificial Sequence
  Antisense Therapy ODNs +5 to +27
taacatagcagaagcatatgatt

SEQ ID NO: 899 - DNA, 20 bp, Artificial Sequence
  Anitsense Therapy ODNs -10 to +10
atgattgcattgtcaaaaac

SEQ ID NO: 900 - DNA, 21 bp, Artificial Sequence
  Sense Control ODNs +1 to +21
atgcaatcatatgcttctgct

SEQ ID NO: 901 - DNA, 23 bp, Artificial Sequence
  Sense Control ODNs +5 to +27
aatcatatgcttctgctatgtta

SEQ ID NO: 902 - DNA, 20 bp, Artificial Sequence
  Sense Control ODNs -10 to +10
gtttttgacaatgcaatcat

SEQ ID NO: 903 - DNA, 897 bp, Artificial Sequence
  cDNA sequence NM 003140.2
gttgagggggtgttgagggcggagaaatgcaagtttcattacaaaagttaacgtaacaaagaatctggtagaagtgagttttggatagtaaaataagtttcgaactctggcacctttcaattttgtcgcactctccttgtttttgacaatgcaatcatatgcttctgctatgttaagcgtattcaacagcgatgattacagtccagctgtgcaagagaatattcccgctctccggagaagctcttccttcctttgcactgaaagctgtaactctaagtatcagtgtgaaacgggagaaaacagtaaaggcaacgtccaggatagagtgaagcgacccatgaacgcattcatcgtgtggtctcgcgatcagaggcgcaagatggctctagagaatcccagaatgcgaaactcagagatcagcaagcagctgggataccagtggaaaatgcttactgaagccgaaaaatggccattcttccaggaggcacagaaattacaggccatgcacagagagaaatacccgaattataagtatcgacctcgtcggaaggcgaagatgctgccgaagaattgcagtttgcttcccgcagatcccgcttcggtactctgcagcgaagtgcaactggacaacaggttgtacagggatgactgtacgaaagccacacactcaagaatggagcaccagctaggccacttaccgcccatcaacgcagccagctcaccgcagcaacgggaccgctacagccactggacaaagctgtaggacaatcgggtaacattggctacaaagacctacctagatgctcctttttacgataacttacagccctcactttcttatgtttagtttcaatattgttttcttttctctggctaataaaggccttattcatttca

SEQ ID NO: 904 - DNA, 21 bp, Artificial Sequence
  Antisense ODN1
gcgcttgacatggccctccat

SEQ ID NO: 905 - DNA, 22 bp, Artificial Sequence
  Antisense ODN2
catggggcgcttgacatggccc

SEQ ID NO: 906 - DNA, 20 bp, Artificial Sequence
  antisense ODN3
ggccctccatgctatctaga

SEQ ID NO: 907 - DNA, 21 bp, Artificial Sequence
  Control (sense) ODN1
atggagggccatgtcaagcgc

SEQ ID NO: 908 - DNA, 22 bp, Artificial Sequence
  Control (sense) ODN2
agggccatgtcaagcgccccat

SEQ ID NO: 909 - DNA, 20 bp, Artificial Sequence
  Control (sense) ODN3
tctagatagcatggagggcc

SEQ ID NO: 910 - DNA, 615 bp, Artificial Sequence
  cDNA encoding SRY
atgcaatcatatgcttctgctatgttaagcgtattcaacagcgatgattacagtccagctgtgcaagagaatattcccgctctccggagaagctcttccttcctttgcactgaaagctgtaactctaagtatcagtgtgaaacgggagaaaacagtaaaggcaacgtccaggatagagtgaagcgacccatgaacgcattcatcgtgtggtctcgcgatcacaggcgcaagatggctctagagaatcccagaatgcgaaactcagagatcagcaagcagctgggataccagtggaaaatgcttactgaagccgaaaaatggccattcttccaggaggcacagaaattacagcccatgcacagagagaaatacccgaattataagtatcgacctcgtcggaaggcgaagatgctgccgaagaattgcagtttgcttcccgcagatcccgcttcggtactctgcagcgaagtgcaactggacaacaggttgtacagggatgactgtacgaaagccacacactcaagaatggagcaccagctaggccacttaccgcccatcaacgcagccagctcaccgcagcaacgggaccgctacagccactggacaaagctgtag

SEQ ID NO: 911 - protein, 204 aa, Artificial Sequence
  Amino acid sequence of SRY
MQSYASAMLSVFNSDDYSPAVQENIPALRRSSSFLCTESCNSKYQCETGENSKGNVQDRVKRPMNAFIVWSRDQRRKMALENPRMRNSEISKQLGYQWKMLTEAEKWPFFQEAQKLQAMHREKYPNYKYRPRRKAKMLPKNCSLLPADPASVLCSEVQLDNRLYRDDCTKATHSRMEHQLGHLPPINAASSPQQRDRYSHWTKL

(19) United States
(12) Patent Application Publication
(10) Pub. No.: US 2018/0092992 A1
(43) Pub. Date: Apr. 5, 2018

(54) METHOD OF TREATMENT

(71) Applicant: Hudson Institute of Medical Research, Clayton, Victoria (AU)

(72) Inventors: Vincent HARLEY, Malvern (AU); Joohyung LEE, Balwyn (AU)

(21) Appl. No.: 15/566,178

(22) PCT Filed: Apr. 14, 2016
(86) PCT No.: PCT/AU2016/050272

§ 371 Date: Oct. 12, 2017

(30) Foreign Application Priority Data
Apr. 15, 2015 (AU) 2015901338

(51) Int. Cl. A61K 48/00; C12N 15/113; A61P 25/18; C12N 15/63

(52) U.S. Cl. CPC A61K 48/0066; C12N 15/113; A61P 25/18; C12N 15/63; C12N 2310/531; C12N 2310/11; C12N 2310/20; C12N 2320/31; C12N 2310/315; C12Q 2600/178

(57) ABSTRACT

The present specification teaches generally a method for the treatment or prophylaxis of a male-biased neurological disorder in male subjects.

### FILING DATA

This application is associated with and claims priority from Australian Provisional Patent Application No. 2015901338, filed on 15 Apr. 2015, entitled “A method of treatment”, the entire contents of which, are incorporated herein by reference.

### BACKGROUND

### Field

The present specification teaches generally a method for the treatment or prophylaxis of a male-biased neurological disorder in male subjects.

### Description of Related Art

Bibliographic details of the publications referred to by author in this specification are collected alphabetically at the end of the description.

Reference to any prior art in this specification is not, and should not be taken as, an acknowledgment or any form of suggestion that this prior art forms part of the common general knowledge in any country.

The primary event of sexual development in mammals is the development of the gonadal sex from a bipotential and undifferentiated gonad into either testes or ovaries. This process, known as sex determination, is triggered by the SRY gene (Sex-determining Region, Y chromosome). Evidence that SRY was sex determining initially came from the microinjection of a 14.6 kb genomic DNA sequence containing the mouse SRY gene into chromosomally female embryos. The resulting transgenic mice developed phenotypically as males (Koopman et al. (1991) Nature 351(6322):117-21). SRY belongs to the Sox (SRY-box) family, whose members are characterized by a common HMG (high mobility group) DNA-binding motif (Laudet et al. (1993) Nucleic Acids Res, 21(10) 2493-501; Wegner (1999) Nucleic Acids Res, 27(6):1409-20). Sox genes have been documented in a wide range of developmental processes, including neurogenesis (Sox2, 3 and 10) [Hargrave et al. (1997) Dev Dyn, 210(2):79-86; Rex et al. (1997) Mech Dev, 66(1-2):39-53; Uwanogho et al. (1995) Mech Dev, 49(1-2):23-36] and sex determination (Sox9). In addition, mutational analysis has suggested a role for Sox genes in influencing cell fate decisions during development (Pevny and Lovell-Badge (1997) Curr Opin Genet Dev. 7(3):338-44). Encoding a 204 amino acid protein, SRY is thought to bind and sharply bend DNA by means of its HMG box to regulate male-specific gene expression (Ferrari et al. (1992) Embo J, 11(12):4497-506; Harley et al. (1992) Science, 255(5043):453-6; King and Weiss (1993) Proc Natl Acad Sci USA, 90(24):11990-4; Nasrin et al. (1991) Nature, 354(6351):317-20). The transient expression of SRY during a brief period in the developing genital ridge, between embryonic days E10.5 and E12.5, is what triggers testis development from a bipotential gonad (Koopman et al. (1990) Nature, 348(6300):450-2). After this strictly regulated window of expression in mouse fetal gonads, SRY is re-expressed in the adult testis.

The substantia nigra (SN) is a nucleus located in the midbrain that plays a pivotal role in the control of voluntary movement. The SN is cytoarchitecturally divided into three different parts: the SN pars compacta (SNc), the SN pars reticulata, and the SN pars lateralis (Olanow and Tatton (1999) Annu Rev Neurosci, 22:123-44). The SNc, a region rich in dopaminergic neurons, has been associated with the neurological disorder, Parkinson's disease, as dopaminergic neurons of the SNc preferentially degenerate in Parkinson's disease patients (Castillo et al. (1998) Mol Cell Neurosci, 11(1-2):36-46). Parkinson's disease is a neurodegenerative disorder caused by SNc dopaminergic cell death and characterized by rigidity, rest tremor, postural instability and bradykinesia. Dopaminergic neurons of the SNc regulate motor function via nigrostriatal projections to the dorsolateral striatum. Transcriptional factors such as β-catenin, Nurr1 and Pitx3 control the dopamine phenotype (Maxwell et al. (2005) Dev Biol, 282(2):467-79; Malbon (2004) Front Biosci, 9:1048-58).

Many gender differences in the function of the SNc and its striatal projections have been described (Saunders-Pullman (2003) Endocrine, 21(1):81-7). The clinical implications of these differences are apparent in the onset and progression of Parkinson's disease. Males are more susceptible to Parkinson's disease than females.

Neurological degenerative diseases are a significant issue in an aging population. The social and medical cost of managing human subjects afflicted with such disorders places an enormous strain on economies around the world. New therapeutic protocols to ameliorate the devastating impact of neurodegenerative disorders are urgently needed. It had been deomonstrated that SRY down-regulation in the SNc impaired motor function in male rats (Dewing et al. (2006) Current Biology 16(4):415-420). This led to strategies to facilitate elevating levels of SRY. In accordance with the present invention, it is proposed that the reverse of this approach is in fact required.

### SUMMARY

Nucleotide and amino acid sequences are referred to by a sequence identifier number (SEQ ID NO). The SEQ ID NOs correspond numerically to the sequence identifiers <400>1 (SEQ ID NO:1), <400>2 (SEQ ID NO:2), etc. A summary of the sequence identifiers is provided in Table 1. A sequence listing is provided after the claims.

A summary of sequence identifiers used throughout the subject specification is provided in Table 1.

The present specification teaches that sex differences in the molecular characteristics of brain regions and their associated behavior are influenced by genetic factors independently of gonadal hormones. It is proposed herein that SRY, the key male specific gene, is expressed in male dopamine neurons where it regulates dopamine synthesis and motor function. Certain neurological disorders develop or are otherwise exacerbated by loss of dopamine-producing cells. Contemplated herein is a method of ameliorating symptoms of male-biased neurological disorders or mitigating the severity of these disorders by specifically down-regulating expression or activity or function of SRY in dopaminergic nerve cells in male subjects. It is proposed herein that following trauma, injury, disease or exposure to toxins or toxicants, SRY levels are at least initially elevated in dopaminergic nerve cells and this leads to loss of dopamine-producing cells. Hence, male-biased neurological disorders are proposed herein to be treated by selectively down-regulating levels of functional or active SRY protein or down-regulating expression of the gene encoding SRY in nerve cells such as dopaminergic neurons. Agents which act as antagonists of SRY or SRY gene expression are neuroprotective in male subjects such as human male subjects.

Male-biased neurological disorders contemplated herein include but are not limited to Parkinson's disease, autism, epilepsy, attention deficit hyperactivity disorder (ADHD), psychosis including schizophrenia, drug addiction and pain. Furthermore, any disorder associated with loss of dopamine-producing cells in males is encompassed by the present invention.

Provided herein is a medical protocol to treat a male-biased neurological disorder by the down-regulation of expression or activity of SRY in a nerve cell such as a dopaminergic neurons in male subjects alone or together with another treatment or behavioral modification. Pharmaceutical compositions, medicaments and treatment kits are also taught herein.

The present specification teaches a method for the treatment or prophylaxis of a male-biased neurological disorder in a male subject, the method comprising administering to the male, an agent or vehicle carrying the agent which enters the brain and inhibits expression of the gene encoding sex-determining region, Y chromosome (SRY) or inhibits SRY function in a dopamine producing nerve cell in an amount effective to ameliorate symptoms, prevent development of the symptoms or minimize further progression of the symptoms of the neurological disorder.

Further taught herein is a therapeutic protocol for treating or preventing a male-biased neurological disorder in a male subject, the protocol comprising:

(i) identifying and selecting the male subject based on behavior, genetic predisposition, symptoms or age or exposure to toxins or toxicants;

(ii) administering to the male, an agent or vehicle carrying the agent which enters the brain and inhibits expression of the gene encoding sex-determining region, Y chromosome (SRY) or inhibits SRY function in a dopamine producing nerve cell in an amount effective to ameliorate symptoms, prevent development of the symptoms or minimize further progression of the symptoms of the neurological disorder;

(iii) monitoring the symptoms and behavior of the male subject;

(iv) provide further agent or other medicaments or behavioral modification as required to maintain the health of the male subject.

Enabled herein is the use of an agent which antagonizes SRY activity or function or SRY gene expression in the manufacture of a medicament to treat or ameliorate the symptoms of a male-biased neurological disorder in a male subject. Examples of antisense oligonucleotides for human use include SEQ ID NOs:897 through 899 and SEQ ID NOs:888 (ASO-A), 890 (ASO-B), 894 (ASO-D) and 896 (ASO-E).

A list of abbreviations used throughout the subject specification are provided in Table 2.

TABLE 1
Summary of sequence identifiers

| SEQUENCE | |
|---|---|
| ID NO: | DESCRIPTION |
| 1 to 886 | Potential/targe sites for down-regulation of human SrYR |
| | gene |
| 887 | Target Site A (human) |
| 888 | ASO-A (human) |
| 889 | Target Site B (human) |
| 890 | ASO-B (human) |
| 891 | Target Site C (human) |
| 892 | ASO-C (human) |
| 893 | Target Site D (human) |
| 894 | ASO-D (human) |
| 895 | Target Site E (human) |
| 896 | ASO-E (human) |
| 897 | Antisense Therapy ODNs +1 to +21 (human) |
| 898 | Antisense Therapy ODNs +5 to +27 (human) |
| 899 | Antisense Therapy ODNs −10 to +10 (human) |
| 900 | Sense Control ODNs +1 to +21 (human) |
| 901 | Sense Control ODNs +5 to +27 (human) |
| 902 | Sense Control ODNs −10 to +10 (human) |
| 903 | Nucleotide sequence of cDNA encoding human |
| | SRY: NM 003140. [symbol] |
| 904 | Antisense ODN 1 (rat) |
| 905 | Antisense ODN 2 (rat) |
| 906 | Antisense ODN 3 (rat) |
| 907 | Control (sense) ODN 1 (rat) |
| 908 | Control (sense) ODN 2 (rat) |
| 909 | Control (sense) ODN 3 (rat) |
| 910 | cDNA encoding rat SRY |
| 911 | Amino acid sequence of rat SRY |

[symbol]

indicates data missing or illegible when filed

TABLE 2
Abbreviations

| ABBREVIATION | DESCRIPTION |
|---|---|
| 6-OHDA | 6-Hydroxydopamine hydrobromide |
| CRISPR DNA | Clustered regularly interspaced short palindromic |
| | repeat DNA |
| D2R | Dopamine receptor D2 |
| DA | Dopamine |
| DBH | Dopamine-β-hydroxylase |
| DDC | DOPA decarboxylase |
| D-DOPA | D-3,4-dihydroxyphenylalanine |
| DOPAC | Dihydroxyphenylacetic acid |
| HMG | High mobility group |
| L-DOPA | L-3,4-dihydroxyphenylalanine |
| MAO-A | Monamine oxidase-A |
| ODN | Oligodeoxynucleotide |
| ORN | Oligoribonucleotide |
| SN | Substantia nigra |
| SNc | Substantia nigra pars compacta |
| SRY | Sex-determining region, Y chromosome |
| Target Site A | Translation start site of SRY |
| Target Site E | Poly A signal of SRY |
| Target Sites B to D | mRNA loop of SRY |
| TH | Tyrosine hydroxylase |

### BRIEF DESCRIPTION OF THE FIGURES

FIGS. 1 through D are graphical representations showing that SRY controls motor function and nigrostriatal dopamine levels in male rats A) Effect of repeated nigral SRY antisense or sense ODN injections (2 μg/daily for 10 days) on motor function in male or female rats. B) Motor function was assessed by the limb-use asymmetry (top) and rotarod (below) tests in male (left) or female (right) rats. Following the last behavioural test, brains were processed for C) nigral SRY, Sox-6, Sox-3, TH, DDC, MAO A, and D2R mRNA or D) striatal DA and DOPAC measurements (n≧10/group; *P<0.05 compared to sense-treated group; # P<0.05 compared to day 0).

FIGS. 2A and B are graphical and photographical representations showing the repeated SRY antisense treatment, before 6-OHDA injection, attenuates 6-OHDA-induced motor deficits and nigral DA cell loss in male rats. Effect of repeated nigral SRY antisense or sense ODN treatment (2 μg/daily, 10 days) on 6-OHDA induced motor deficits and dopamine cell loss in male rats. A) Motor function was assessed by the limb-use asymmetry (left) and amphetamine-induced rotation (right) tests B) At the end of the motor behavioural tests, the brains were processed and nigral dopamine cell counts was determined (n=20/group; *P<0.05 **P<0.01 compared to sense-treated group; # P<0.05 compared to day 0; ̂P<0.05 compared to day 10).

FIGS. 3A and B are graphical and photographic representations showing the repeated SRY antisense treatment, following 6-OHDA injection, attenuates 6-OHDA-induced motor deficits and nigral. DA cell loss in male rats. Effect of repeated nigral SRY antisense or sense ODN treatment (2 μg/daily, 10 days) on 6-OHDA induced motor deficits and dopamine cell loss in male rats. A) Motor function was assessed by the limb-use asymmetry (left) and amphetamine-induced rotation (right) tests B) At the end of the motor behavioural tests, the brains were processed and nigral dopamine cell counts was determined (n=10/group *P<0.05 **P<0.01 compared to sense-treated group; # P<0.05 compared to day 0; ̂P<0.05 compared to day 10).

FIGS. 4A and B are graphical and photographical representations showing the repeated SRY antisense treatment, before rotenone injection, attenuates rotenone-induced motor deficits and nigral. DA cell loss in male rats. Effect of repeated nigral SRY antisense or sense ODN treatment (2 μg/daily, 10 days) on rotenone-induced motor deficits and dopamine cell loss in male rats. A) Motor function was assessed by the limb-use asymmetry (left) and amphetamine-induced rotation (right) tests B) At the end of the motor behavioural tests, the brains were processed and nigral dopamine cell counts was determined (n=10/group *P<0.05 **P<0.01 compared to sense-treated group; # P<0.05 compared to day 0; ̂P<0.05 compared to day 10).

FIGS. 5A and B are graphical and photographical representations showing the repeated SRY antisense treatment, before 6-OHDA injection, does not affect 6-OHDA-induced motor deficits and nigral DA cell loss in female rats. Effect of repeated nigral SRY antisense or sense ODN treatment (2 μg/daily, 10 days) on 6-OHDA-induced motor deficits and dopamine cell loss in female rats. A) Motor function was assessed by the limb-use asymmetry (left) and amphetamine-induced rotation (right) tests B) At the end of the motor behavioural tests, the brains were processed and nigral dopamine cell counts was determined (n=10/group; ̂P<0.05 compared to day 10.

### DETAILED DESCRIPTION

Throughout this specification, unless the context requires otherwise, the word “comprise” or variations such as “comprises” or “comprising”, will be understood to imply the inclusion of a stated element or integer or method step or group of elements or integers or method steps but not the exclusion of any element or integer or method step or group of elements or integers or method steps.

As used in the subject specification, the singular forms “a”, “an” and “the” include plural aspects unless the context clearly dictates otherwise. Thus, for example, reference to “a neuron” includes a single neuron, as well as two or more neurons; reference to “an agent” includes a single agent, as well as two or more agents; reference to “the disclosure” includes single and multiple aspects taught by the disclosure; and so forth. Aspects taught and enabled herein are encompassed by the term “invention”. All such aspects are enabled within the width of the claimed invention.

The present invention relates to a method for the treatment or prophylaxis of a male-biased neurological condition in a male subject. The method is predicated in part on the determination that following a neurological environment resulting from damage, injury, toxicity, disease or infection, sex-determining region, Y chromosome (SRY) gene expression in male dopamine-producing nerve cells cause elevated dopamine synthesis. Whilst this may be initially protective, it ultimately leads to selective loss of dopamine-producing cells. Hence, down-regulating levels of SRY or its ability to function or down-regulating expression of the gene encoding SRY, is neuroprotective. In particular, dopaminergic neurons are the target for down-regulation of SRY function, activity or gene expression.

Accordingly, enabled herein is a method for the treatment or prophylaxis of a male-biased neurological disorder in a male subject, the method comprising administering to the male subject, an agent or vehicle carrying the agent which enters the brain and inhibits expression of the gene encoding sex-determining region, Y chromosome (SRY) or inhibits SRY function or activity in a dopamine-producing nerve cell in an amount effective to ameliorate symptoms, prevent development of the symptoms or minimize further progression of the symptoms of the neurological disorder.

Reference to a “dopamine-producing nerve cell” includes a dopamine-producing neuron which is also referred to as a dopaminergic neuron. The dopaminergic neuron is generally located in the substantia nigra pars compacta (SNc) of the brain. Notwithstanding, for various neurological disorders, it may be necessary to target other areas of the brain such as the substantia nigra (SN) pars reticulata and SN pars lateralis and all such areas are encompassed by the present invention. It is proposed herein that down-regulation of SRY function, activity or levels reduces or inhibits progressive loss of dopamine-producing cells. Reference to reducing dopamine-producing cell loss includes, taking the percentage of dopamine-producing cells in a normal brain as 100%, that the male subject being treated maintains from at least about 40% to 100% dopamine-producing cells, or a percentage inbetween, such as 50%, 60%, 70%, 80% or 90%. A “normal brain” includes a brain from an asymptomatic subject.

It is proposed herein that the neurological disorder is a male-biased condition and includes a disorder associated with loss of dopamine-producing cells. Such disorders include Parkinson's disease (PD), autism, epilepsy, attention deficit hyperactivity disorder (ADHD), a psychosis, drug addiction and pain. Reference to a “psychosis” includes schizophrenia. Any disorder associated with changes in dopamine levels or any type of dopamine dysfunction or dysregulation are encompassed by the present invention including behavioral disorders exhibiting reward characteristics leading to potentially anti-social or self-harm activities.

In an embodiment, the neurological disorder is Parkinson's disease.

Accordingly, taught herein is a method for the treatment or prophylaxis of Parkinson's disease in a male subject, the method comprising administering to the male subject, an agent or vehicle carrying the agent which enters the brain and inhibits expression of the gene encoding SRY or inhibits SRY function or activity in a dopamine-producing nerve cell in an amount effective to ameliorate symptoms, prevent development of the symptoms or minimize further progression of the symptoms of Parkinson's disease.

In another embodiment, the neurological disorder is ADHD.

Hence, the present specification is instructional for a method for the treatment or prophylaxis of ADHD in a male subject, the method comprising administering to the male subject, an agent or vehicle carrying the agent which enters the brain and inhibits expression of the gene encoding SRY or inhibits SRY function or activity in a dopamine-producing nerve cell in an amount effective to ameliorate symptoms, prevent development of the symptoms or minimize further progression of the symptoms of the ADHD.

In yet another embodiment, the neurological disorder is autism.

Enabled herein is a method for the treatment or prophylaxis of autism in a male subject, the method comprising administering to the male subject, an agent or vehicle carrying the agent which enters the brain and inhibits expression of the gene encoding SRY or inhibits SRY function or activity in a dopamine-producing nerve cell in an amount effective to ameliorate symptoms, prevent development of the symptoms or minimize further progression of the symptoms of the autism.

Still in yet another embodiment, the neurological disorder is epilepsy.

Hence, taught herein is method for the treatment or prophylaxis of epilepsy in a male subject, the method comprising administering to the male subject, an agent or vehicle carrying the agent which enters the brain and inhibits expression of the gene encoding SRY or inhibits SRY function or activity in a dopamine-producing nerve cell in an amount effective to ameliorate symptoms, prevent development of the symptoms or minimize further progression of the symptoms of epilepsy.

Reference to “dopamine-producing nerve cell” includes a dopaminergic neuron.

An agent is contemplated herein which:

down-regulates expression of the gene encoding SRY;

(ii) inhibits or reduces the function of SRY;

(iii) inhibits or reduces the activity of SRY; and/or

(iv) inhibits or reduces the function or level of a component in a signaling pathway associated with SRY.

The agent is in effect an antagonist of SRY function, activity or gene expression. The agent may be referred to as an antagonist, medicament, pharmaceutical, active amongst other terms. The agent encompasses nucleic acids, nucleic acid-based constructs (including phosphorothioated nucleic acids and CRISPR/Cas nucleic acids), proteins and small chemical molecules.

By “down-regulate” expression of the gene encoding SRY is meant preventing or reducing transcription or translation of the gene. In an embodiment, the SRY mRNA is targeted by an antisense or sense oligonucleotide. Any portion of the mRNA or DNA sequence may be targeted. Examples of DNA target sites on the SRY gene comprise any target site on SEQ ID NO:903 or its mRNA equivalent or a regulatory region such as a promoter region or polyadenylation signal. Examples include comprising DNA sequences selected from SEQ ID NOs:1 through 886 or an mRNA equivalent. Particular examples include target sites SEQ ID NOs:887, 889, 891, 893 and 895 (e.g. antisense molecules SEQ ID NOs:888, 890, 892, 894 and 896, respectively) and antisense molecules SEQ ID NOs:897, 898 and 899. The present specification contemplates any nucleic acid molecule comprising from about 6 to about 1,000 nucleotides which is capable of hybridizing to SRY mRNA transcript under low stringency conditions, or medium stringency conditions or high stringency conditions and prevent translation of the SRY mRNA transcript or at least reduce the amount of translation to thereby reduce active SRY levels. Target sites at the non-coding 5′ and 3′ ends of the gene may also be targeted. Examples include from 10 nucleotides in length to 100 nucleotides in length. In an embodiment, the ODN comprises 20 nucleotides.

Reference herein to a low stringency includes and encompasses from at least about 0 to at least about 15% v/v formamide and from at least about 1M to at least about 2M salt for hybridization, and at least about 1M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5M to at least about 0.9M salt for hybridization, and at least about 0.5M to at least about 0.9 M salt for washing conditions, or high stringency, which includes and encompasses from at least about 31% v/v to at least about 50% v/v formamide and from at least about 0.01 M to at least about 0.15M salt for hybridization, and at least about 0.01M to at least about 0.15M salt for washing conditions. In general, washing is carried out Tₘ=69.3+0.41 (G+C) % (Marmur and Doty, J. Mol. Biol. 5: 109, 1962). However, the Tₘ of a duplex DNA decreases by 1° C. with every increase of 1% in the number of mismatch base pairs (Bonner and Laskey (1974) Eur. J. Biochem. 46: 83). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The term “oligonucleotide” includes an oligodeoxynucleotide (ODN) or a ribonucleotide (ORN) which may be antisense to the coding sequence or sense to it. The term generally refers to a plurality of linked nucleoside units.

Such oligonucleotides can be obtained from existing nucleic acid sources, including genomic or cDNA or mRNA or produced by synthetic methods. In exemplarily embodiments, each nucleoside unit can encompass various chemical modifications and substitutions as compared to wild-type oligonucleotides, including but not limited to modified nucleoside base and/or modified sugar unit. Examples of chemical modifications are known to the person skilled in the art and are described, for example, in Uhlmann et al. (1990) Chem. Rev. 90:543; Hunziker. et al. (1995) Mod. Syn. Methods 7:331-417; and Crooke et al. (1996) Ann. Rev. Pharm. Tox. 36:107-129. The nucleoside residues can be coupled to each other by any of the numerous known internucleoside linkages. Such internucleoside linkages include, without limitation, phosphodiester, phosphorothioate, phosphorodithioate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone intemucleoside linkages. The term “oligonucleotide” also encompasses polynucleosides having one or more stereospecific intemucleoside linkage (e.g. phosphorothioate, alkylphosphonate, or phosphotriester linkages). As used herein, the term “oligonucleotide” includes polynucleosides, having any such internucleoside linkage, whether or not the linkage comprises a phosphate group. In an embodiment, these internucleoside linkages may be phosphodiester, phosphorothioate or phosphorodithioate linkages, or combinations thereof.

The nucleosides may be 2′-substituted. The term “2′-substituted” generally includes nucleosides in which the hydroxyl group at the 2′ position of the pentose moiety is substituted to produce a 2′-substituted or 2′-O-substituted nucleoside. In an embodiment, such substitution is with a lower hydrocarbyl group containing 1-6 saturated or unsaturated carbon atoms, with a halogen atom, or with an aryl group having 6-10 carbon atoms, wherein such hydrocarbyl, or aryl group may be unsubstituted or may be substituted, for example, but not limited to substitution with halo, hydroxy, trifluoromethyl, cyano, nitro, acyl, acyloxy, alkoxy, carboxyl, carboalkoxy or amino groups. Examples of 2′-O-substituted nucleosides include, without limitation 2′-amino, 2′-fluoro, 2′-allyl, 2′-O-alkyl and 2′-propargyl ribonucleosides or arabinosides, 2′-O-methylribonucleosides or 2′-O-methylarabinosides and 2′-O-methoxyethoxyribonucleosides or 2′-O-methoxyethoxyarabinosides.

The term “about” generally means that the exact number is not critical. Thus, the number of from about 6 to about 1,000 nucleoside residues in an oligonucleotide according to this aspect of the present invention is not necessarily critical, and oligonucleotides having fewer or more nucleoside residues, or from one to several, fewer or additional nucleoside residues are contemplated as equivalents of each of the embodiments described above. Oligonucleotides which target non-coding 5′ and 3′ ends of the SRY mRNA transcript or corresponding portion in the gene are also contemplated herein.

The term “antisense oligonucleotide” generally refers to strands of DNA or RNA or combinations thereof that are complementary to a chosen nucleic acid sequence such as mRNA transcribed from the SRY gene. In an embodiment the target nucleic acid is to SRY mRNA transcript. When introduced into a nerve cell, an antisense oligonucleotide can bind to and cause the reduction in the translation of SRY RNA to which it is complementary. If binding takes places, this nucleic acid complex can be degraded by endogenous enzymes. Antisense oligonucleotides include, but are not limited to, traditional antisense oligonucleotides but also include short interfering RNA (siRNA), micro RNA (mRNA), single stranded RNAs, hairpin RNAs and ribozymes, and deoxyribonucleotide equivalents of any of these.

Useful oligonucleotides include clustered regularly interspaced short palindromic repeat (CRISPR) DNAs. These are DNA loci comprising short repetitions of nucleotide sequences interspersed with spacer DNA. CRISPRs in association with Cas genes, are used for gene editing by the insertion, deletion or substitution of target nucleotide sequences in coding, non-coding and regulatory regions. For example, CRISPRs can deliver the Cas9 endonuclease into a cell using guide RNAs (Wang et al. (2013) Cell 153(4):910-918).

Hence, enabled herein, is a method for the treatment or prophylaxis of a male-based neurological disorder in a male subject, the method comprising administering to the male subject, a CRISPR/Cas agent which enters the brain and disrupts the SRY gene thereby reducing its ability to express a functional protein in a dopaminergic nerve cell. The amount of CRISPR/Cas agent is effective to ameliorate symptoms or prevent development of symptoms or minimize further progression of symptoms of the neurological disease.

Expression vectors comprising nucleic acid molecules may encode a sense or antisense oligonucleotide or a protein antagonist of SRY. These may be present in a virus or viroid particular for use to introduce to a target nerve cell in the brain. The nucleic acid is operably linked to regulatory elements needed for gene expression. Accordingly, incorporation of the DNA or RNA molecule into a delivery virus or other vehicle results in the expression of the DNA or RNA encoding the oligonucleotide or protein when the virus introduces the expression vector to the nerve cell.

Hence, the nucleic acid molecule that includes the nucleotide sequence encoding the oligonucleotide or protein operably linked to the regulatory elements may be introduced to a target dopaminergic nerve cell via viral vector or agent. Alternatively, linear DNA or RNA which can integrate into the chromosome may be introduced into the target cell. When introducing DNA or RNA into a cell, reagents which promote DNA or RNA integration into chromosomes or transcriptome may be added.

The necessary elements of an expression vector include a nucleotide sequence that encodes the oligonucleotide or a protein antagonist of SRY and the regulatory elements necessary for expression of that sequence in the target nerve cells. The regulatory elements are operably linked to the nucleotide sequence that encodes the oligonucleotide or protein antagonist to enable expression inside the target nerve cell. The nucleotide sequence may be cDNA, genomic DNA, synthesized DNA or a hybrid thereof or an RNA molecule such as mRNA.

The regulatory elements necessary for gene expression include: a promoter, an initiation codon, a stop codon, and a polyadenylation signal. It is necessary that these elements be operable in the dopaminergic neurons. Moreover, it is necessary that these elements be operably linked to the nucleotide sequence that encodes the oligonucleotide or protein antagonist such that the nucleotide sequence can be expressed in the nerve cells and thus the oligonucleotide or protein antagonist can be produced. Reference to nerve cell includes a dopaminergic neuron.

Other agents contemplated herein include small chemical molecules, antibodies modified to cross the blood brain barrier or introduced directly into the brain and enter neurons, small peptides, cyclohexene derivatives, lipid derivatives, and vehicles used to transport these agents, such as liposomes and genetically modified viral agents which infect target cells and facilitate delivery and expression or production of an agent such as an oligonucleotide.

The term “small molecule” generally refers to small organic compounds that are biologically active. Small molecules may exist naturally or may be created synthetically. Small molecules may include compounds that down-regulate the expression, function or activity of SRY. They may cross the blood brain barrier to be introduced directly to the brain.

Generally, the agents are delivered with a physiologically or pharmaceutically acceptable carrier, diluent or excipient. Hence, formulations, medicaments, therapeutic agents and pharmaceutical compositions comprising the agent and a physiologically or pharmaceutically acceptable carrier, diluent or excipient are contemplated herein.

The term “physiologically acceptable” generally refers to a material that does not interfere with the effectiveness of the agent and that is compatible with a biological system such as a cell, cell culture, tissue, or organism. Generally, the organism is a mammal such as a human.

The term “pharmaceutically acceptable” generally refers to compositions that are suitable for use in humans and animals without undue toxicity.

The term “carrier” generally encompasses any excipient, diluent, filler, salt, buffer, stabilizer, solubilizer, oil, lipid, lipid containing vesicle, microspheres, liposomal encapsulation, or other material well known in the art for use in pharmaceutical formulations. It will be understood that the characteristics of the carrier, excipient, or diluent will depend on the route of administration for a particular application. The preparation of pharmaceutically acceptable formulations containing these materials is described in, e.g. Remington's Pharmaceutical Sciences, 18th Edition, A. Gennaro, ed., Mack Publishing Co., Easton, Pa., 1990.

The terms an “effective amount,” “pharmaceutically effective amount” or “therapeutically effective amount” generally refer to an amount sufficient to affect a desired biological effect, such as beneficial results including reducing dopamine-producing cell loss, reducing SRY function, activity or gene expression or ameliorating or mitigating the effects of neurodegeneration. Thus, an “effective amount” or “sufficient amount” or “pharmaceutically effective amount” or “therapeutically effective-amount” will depend upon the context in which it is being administered. In the context of administering a composition that reduces SRY gene expression or SRY protein function or activity is an amount sufficient to achieve the desired amelioration of neurodegenerative symptoms. Hence, the amount of agent administered is effective to inhibit or reduce the function or level or activity of SRY. Generally, the amount is effective to mitigate the symptoms or underlying cause of the neurological disorder. Hence, in an embodiment, the amount is effective to reduce loss of dopamine-producing cells and to maintain at lest motor function.

The agent may be administered in any way which enables it to reach its target in the brain by any mechanism. Whilst intracranial administration and retrograde transport is contemplated herein, the agent may alternatively need to be administered in a form that can penetrate the blood brain barrier. Hence, the present invention extends to nanobiotechnology-based delivery methods such as passive diffusion, use of lipid or fat soluble substances, active transport using carrier proteins and receptor-mediated transport such as an agent linked to a particular receptor via cerebrospinal fluid following a lumbar puncture. Teaching for neural transplantation include stereotaxic surgery in which an agent formulation is implanted into the brain or grafted into the brain by microsurgery. Techniques for introducing agents have been described (Lindvall et al. (1987) Ann. Neurol. 22:457 468; Madrazo et al. (1987) New Engl. J. Med. 316:831 834; Dewing et al. (2006) supra). Other mechanisms include insertion of a guide cannula to inject the nucleic acid. This include a brain guide cannula such as an MBR style Brain Guide Cannula from BASi (West Lafayette, Ind., USA).

Enabled herein is a therapeutic protocol for treating or preventing a male-biased neurological disorder in a male subject, the protocol comprising:

(i) identifying and selecting the male subject based on behavior, genetic predisposition, symptoms or age or exposure to toxins or toxicants;

(ii) administering to the male subject an agent or vehicle carrying the agent which enters the brain and inhibits expression of the gene encoding SRY or inhibits SRY function or activity in a dopamine-producing nerve cell in an amount effective to ameliorate symptoms, prevent development of the symptoms or minimize further progression of the symptoms of the neurological disorder;

(iii) monitoring the symptoms and behavior of the male subject;

(iv) provide further agent or other medicaments or behavioral modification as required to maintain the health of the male subject.

The dopamine-producing nerve cell includes a dopaminergic neuron.

Further enabled herein is the use of an agent which antagonizes SRY activity or function or SRY gene expression in the manufacture of a medicament to treat or ameliorate the symptoms of a male-biased neurological disorder in a male subject.

The male subject is generally a human male in need of treatment or at risk of requiring treatment, however, non-human mammals may also be treated. This is generally for testing purposes. An example of an “at risk” subject is a person exposed to a toxin or toxicant such as an environmental toxicant or a person genetically predisposed to the condition such as via hereditary means. Hence, the treatment includes potential prevention of symptoms prior to development.

In an embodiment, once treatment is initiated, it will need to be continued for the life of the subject. It is possible that the treatment is only partially effective and results in a slowing down of symptomology. Notwithstanding, the treatment will prolong the quality of life for longer than without treatment.

The agent may be given alone or in combination with other medicaments to assist the patient in mitigating the severity of symptoms or ameliorate the symptoms.

Behavioral modification such as massage, exercise and psychotherapy, and other medicines such as sedatives, anti-epileptic drugs or methylphidale or its derivatives (e.g. Retalin) may also be administered simultaneously or sequentially or independently of the SRY antagonist. Deep brain stimulation (DBS) may also be employed with the SRY antagonist. DBS includes the implantation of a brain pacemaker which emits electrical impulses via implanted electrodes to selected parts of the brain (Kringlebach et al. (2007) Nature Reviews Neuroscience 8:623-635).

Hence, combination therapy is further contemplated herein comprising a first therapeutic protocol comprising administration of an SRY antagonistic agent and one or more other medicaments and/or behavioral modifications.

A therapeutic kit is therefore provided comprising compartments wherein at least one compartment comprises a SRY antagonistic agent and at least one other compartment comprises another medicament useful in the treatment of a neurological condition. The kit may deliver multiple doses for from 2 days to 21 days such as a course lasting 7 days. The kit may also comprise the SRY antagonist alone also designed to dispense multiple doses from 2 days to 21 days. Alternatively, a package is provided with SRY antagonists in pill form which might last for months. The therapeutic kit may further include a medical device such as for deep brain stimulation or a cannula for the brain to permit delivery of the agents.

Dosing will be dependent on the persons, disorder, severity of symptoms and the like. Hence, a single daily dose to multiple daily doses may be required, generally for the life of the male subject.

### EXAMPLES

Aspects disclosed herein are further described by the following non-limiting Examples.

### Materials and Methods

Adult Long-Evans male and female rats weighing between 280 and 350 g were used. Animals were housed in a 12 hour light:dark cycle room and had access to food and water ad libitum.

### Experimental Design

Study 1. The Effect of Reduced Nigral SRY Levels on Motor and Nigrostriatal Function in Normal Rats.

The effect of reduced nigral SRY levels via repeated SRY antisense oligonucleotide (ODN) injections, on motor function was assessed. SRY antisense or sense ODN was injected daily into the right SNc (2 μg) for 10 days in male or female rats. Motor function was assessed by the limb-use asymmetry and rotarod tests prior to (day 0) and at the end of ODN (day 10) treatment. Following the last behavioral test, brains were processed for measurement striatal DA/DOPAC, and nigral mRNA expression.

### Study 2. The Effect of Toxin-Induced Injury on Nigral SRY Expression in Male Rats.

Partial lesion of nigrostriatal dopamine system was achieved by injection of the 6-OHDA or rotenone into the right SNc. Following surgical anesthesia, rats were placed into a stereotaxic frame. Varying amounts of 6-OHDA (15 or 30 μg/μ1), rotenone (30 μg/μ1) or vehicle (0.1 w/v ascorbic acid or 1% w/v DMSO in saline) was injected into the right SNc (bregma, 5.3 mm posterior, 3 mm lateral from bregma, and 6.0 mm ventral to the surface of dura). Following assessment of motor function pre-surgery, motor function was assessed at day 2, 7, 14 or 28 days post-treatment in male rats. At the end of the motor behavior studies at different time points post 6-OHDA treatment, rats were killed and the brains were processed for nigral SRY and tyrosine hydroxylase (TH) mRNA levels.

### Study 3. The Effect of Reduced Nigral SRY Levels on Toxin-Induced Motor Deficits and Dopamine Cell Loss in Normal Rats.

The effect of repeated nigral SRY antisense or sense ODN injections (2 μg/daily for 10 days) on motor function was assessed in male or female rats injected with a single does of 6-OHDA (30 μg/μl) or rotenone (30 μg/μl) into the right SNc. Motor function was assessed by the limb-use asymmetry and amphetamine-induced rotation test in male or female rats. Following the last behavioral test, brains were processed for measurement of nigral mRNA, nigral TH and striatal DAT immunohistochemistry. Rat ODNs are shown in Table 3.

### Surgery and Drug Injections

### Sterotaxic Implantation of Cannula in the Rat SNc

Following surgical anesthesia, the rats were fixed in a stereotaxic frame. Unilateral guide cannula (22GA) directed at the right SNc was implanted at 5.3 mm posterior, 2 mm lateral from bregma, and 6.0 mm ventral to the surface of dura. The guide cannula was secured to the skull with stainless-steel screws and dental cement. Dummy cannulae that protruded <0.5 mm beyond the opening were placed in the guide cannulae.

Chronic Injection of SRY Antisense Oligonucleotide into the Rat SNc

The antisense used for infusions was a cocktail of distinct ODNs (Table 3) added in equal proportions. The first and second ODN, a 21- and 23-mer phosphorothioate-endcapped oligo, were designed to correspond to the rat SRY mRNA (GenBank accession AF274872 [SEQ ID NOs:910 and 911]). The third ODN, a 20-mer, also corresponding to the rat SRY mRNA sequence, was not phosphorothioate-endcapped. The sense triple cocktail ODN corresponded to the complement sequences of the three antisense ODNs. ODNs were HPLC-purified (Invitrogen, Carlsbad, Calif.) and dissolved in artificial cerebrospinal fluid (aCSF) vehicle (0.1M NaCl, 4 mM KCl, 1 mM CaCl₂, 870 mM NaH₂PO₄, and 430 mM MgSO₄ in dH₂O) to a final concentration of 2 μg/μL. Infusions were made through a 22-gauge stainless steel injection cannula, inserted through and extending 0.5 mm below the tip of the indwelling guide cannula, attached with flexible tubing to a 1004, Hamilton syringe mounted on a motorized Harvard micropump (Harvard Apparatus, UK). Infusions were made at a rate of 0.5 μL/minute followed by a 2 minute equilibration period, during which the needle remained in place. All rats were injected unilaterally with antisense or sense ODN daily (2 μg in 1 μL in aCSF) for 10 consecutive days.

TABLE 3
Base sequences and positions of SRY anti-
sense and sense oligodeoxynucleotide

| | | Target | SEQ |
|---|---|---|---|
| | | Region of | ID |
| ODN | Sequence¹ | mRNA | NO: |
| Antisense | GCGCTTGACATGGCCCTCCAT | +1 to +21 | 904 |
| ODN 1 | | | |
| Antisense | CATGGGGCGCTTGACATGGCCC | +5 to +27 | 905 |
| ODN 2 | | | |
| Antisense | GGCCCTCCATGCTATCTAGA | −10 to +10 | 906 |
| ODN 3 | | | |
| Control | ATGGAGGGCCATGTCAAGCGC | +1 to +21 | 907 |
| (sense) | | | |
| ODN 1 | | | |
| Control | AGGGCCATGTCAAGCGCCCCAT | +5 to +27 | 908 |
| (sense) | | | |
| ODN 2 | | | |
| Control | TCTAGATAGCATGGAGGGCC | −10 to +10 | 909 |
| (sense) | | | |
| ODN 3 | | | |

¹Italicized bases are phosphorothioated

Acute Injection of Dopamine Toxins into the Rat SNc

Unilateral lesions of the right SNc were made by acute injection of the dopamine toxins 6-hydroxydopamine hydrobromide (6-OHDA, Sigma-Aldrich, USA) or rotenone (Sigma-Aldrich, USA). Briefly, 6-OHDA (20n) in 1.5 μL 0.1% w/v ascorbic acid saline solution or rotenone (20 μg) in 1.5 μL 1% w/v DMSO/saline solution was infused into the SNc of conscious animals. To avoid degradation, 6-OHDA was prepared fresh for each experiment, kept on ice and protected from light. Infusions were made through a 22-gauge stainless steel injection cannula, inserted through and extending 0.5 mm below the tip of the indwelling guide cannula, attached with flexible tubing to a 100 μL Hamilton syringe mounted on a motorized Harvard micropump (Harvard Apparatus, UK). Infusions were made at a rate of 0.5 μL/minute followed by a 2 minutes equilibration period, during which the needle remained in place.

### Motor Behavioral Tests

Motor functions of rats were assessed using the limb-use asymmetry test, rotarod test and amphetamine-induced rotation test.

### Limb-Use Asymmetry Test

The limb-use asymmetry test assessed spontaneous forelimb usage during vertical explorations in rats, where motor impairment is indicated by a reduction in limb-use contralateral to the site of drug injection. The rat was gently lowered into the cylinder and fore-limb contacts during vertical explorations were video recorded until a total of 30 touches were reached. The data were expressed as the percentage of left (impaired) forepaw contacts; where symmetric paw use (left right) was a measure of unimpaired limb use.

### Rotarod

Rotarod test evaluated balance and motor coordination of rodents by assessing the ability of rodents to stay balanced on a rotating platform. The rats were trained for 2 days prior to the day of the behavioural testing.

### Amphetamine-Induced Rotational Test

The amphetamine-induced rotational test provided a behavioral estimate of dopamine cell death in animals that have received a unilateral 6-OHDA injection (Lee et al. (2008) Brain 131:1574-1587). In brief, rotational behaviour was measured by placing rats in a circular cage where they were tethered to an automated rotometer system and injected with amphetamine (2 mg/kg, intraperitoneally). The total number of rotations was measured for 90 minutes at ten minute intervals. The data are expressed as net rotations per minute, where rotation toward the side of the lesion was given a positive value.

### Isolation of Rat SNc and Striatum

At the end of the behavioral studies, rat brains were either intracardially perfused and processed for immunohistochemistry or isolated fresh and processed for western blot or qRT-PCR. As required, the brains were sectioned using a cryostat (Leica) at −20° C. Serial coronal sections of the striatum and SNc were collected at 16 and 10 μm, respectively. The sections were thaw-mounted onto poly(l-lysine)-coated slides, dried, and stored at −80° C. Between each series a 200 μm slab was collected in order to isolate tissue for RNA and protein processing.

The brains were adjusted on the cryostat until even on the dorsal-ventral and medial-lateral axes. The Paxinos rat brain atlas was used in determining the location of the SNc based on common landmarks such as the ventricles and nerve fibre bundles. Once the SNc was observed, six serial coronal sections were collected followed by the isolation of the SNc from a 200 μm slab to isolate tissue for RNA processing. SNc isolation was achieved by collecting 2×1 mm diameter samples for both the control left SNc and 6-OHDA treated right SNc.

### Immunohistochemistry and Stereology

TH immunohistochemistry was performed by incubating 20 μm-thick SNc sections in sheep anti-TH primary antibody (Pelfreeze, 1:2000, overnight at 4° C.), followed by a biotinylated secondary antibody (goat, anti-sheep IgG, 1:1000, Vector Labs, USA) and reacted with cobalt and nickel-intensified diaminobenzidine (DAB, Sigma-Aldrich). DAB-immunostained sections were counterstained with neutral red. DAT immunohistochemistry was performed by incubating 16 μm-thick striatal sections in rat anti-DAT primary antibody (Chemicon, 1:2000, 78 hrs at 4° C.) followed by a biotinylated secondary antibody (rabbit, anti-rat IgG, 1:500, Vector Labs) and reacted with DAB. DAB-immunostained sections were analysed by bright-field microscopy, using an Olympus microscope equipped with Olympus cellSens image analysis software. TH-immunoreactive and neutral-red positive cell bodies or DAT-immunoreactive terminals were quantified stereologically on regularly spaced sections covering the whole SNc or striatum. The fractionator design for estimating the number of TH-immunoreactive neurons and the number of striatal DAT-immunoreactive axonal varicosities were performed.

Measurement of Rat Nigral mRNA

Due to initial low yields of RNA effort was taken to ensure an RNase and DNase free environment throughout the extraction process, minimizing potential RNA degradation. To ensure an RNase free environment extraction was performed in a fume hood treated with 0.1% w/v SDS followed by 0.1% w/v DEPC-treated water. Pipettes and homogenizing equipment were both thoroughly washed with 0.1% w/v DEPC treated water, followed by 0.1% w/v SDS and again with 0.1% w/v DEPC-treated water. Pestles were washed after each use and handled with tweezers. Only RNase and DNase certified free tips were used. Finally, fresh aliquots of all solutions were used for each batch of RNA extraction. Total RNA was isolated from tissue samples using conditions outlined in the manufacturer's instructions for TRIzol (Registered Trade Mark) Reagent (LifeTechnologies). Briefly, tissue isolated from the Substantia Nigra, as per section 2.3.6, was homogenized in 800 μl of TRIzol (Registered Trade Mark) reagent for 5 minutes at room temperature. Homogenized samples were left to incubate at room temperature for 3 minutes to ensure complete dissociation of nucleoprotein complexes. Samples were then phase separated with the addition of 200 μl of chloroform, shaken vigorously for 15 seconds and spun at 12,000 rpm for 15 minutes at 4° C. Taking care to not disturb lower DNA and organic phases, the top RNA aqueous phase (˜60% of total volume) was transferred to a new tube. A 10 μg volume of RNA free glycogen was then added as a carrier molecule to increase the RNA yield, followed by 400 μl of isopropanol. The sample was then spun at 13,000 rpm for 10 minutes at 4° C. The resulting pellet was washed once with 75% v/v ethanol-0.1% w/v DEPC treated water, before being spun at 7,500 rpm for 10 minutes at 4° C. The pellet was left to dry briefly before being re-dissolved in 20 μl of 0.1% w/v DEPC treated water. RNA quality was checked on 0.75% w/v agarose gel and was analyzed for quality and quantity assessment Nanodrop spectrophotometry at 260 nm (ThermoScientific).

To convert mRNA into cDNA, the appropriate amount (50 ng, 100 ng or 200 ng) of total isolated RNA was reverse transcribed using the modifying enzyme Superscript III (Invitrogen) in 5× First Strand Buffer according to manufacturers specifications. 2 μl of Oligo d(T)15 primer (Roche) was used to reverse transcribe mRNA transcripts in each RT-PCR reaction. Real time quantification of mRNA levels was conducted using the 7900HT Fast Real-Time PCR System (Applied Biosystems) as per manufacturer's instructions. Standard curves were produced by amplification of DNA (serial dilutions) from the gene of interest. Standard curves were used as a reference to establish the amount of mRNA amplified from samples. Values obtained for each sample were standardized to amplification levels of the house keeping genes GAPDH, Tbp-1 and HRPT1. Real time quantification of mRNA levels was conducted using the 7900HT Fast Real-Time PCR System (Applied Biosystems) as per manufacturer's instructions. Using cDNA from M17 cells as a template each gene of interest was PCR amplified, gel purified and serially diluted to generate a set of standards. Standard curves were produced by amplification of DNA (serial dilutions) from the gene of interest. Standard curves were used as a reference to establish the amount of mRNA amplified from samples. Values obtained for each sample were standardized to amplification levels of the house keeping gene GAPDH.

SRY, TH, GADD45γ and GAPDH primers were used to determine the gene transcript levels in response to 6-OHDA treatment. A total volume of 22 μl of PCR mix per sample was made up adding 4 ul of cDNA and 0.41 Taq polymerase to a mixture containing: 10× Taq Buffer MgCl₂ (2 mM), Primer Forward (5 μM), Primer Reverse (5 μM), RNase-free water, dNTP 2.5 mM.

### Statistical Analysis

All values are expressed as the mean±S.E.M. All data were analyzed using tools within Graphpad Prism 5. Motor function of the treatment groups across the days of testing will be analyzed by two-way analysis of variance (ANOVA), with day as the repeated measure factor. Significant differences between the groups in histology or biochemical assays was determined by performing a one-way repeated-measure ANOVA. Bonferroni's post hoc tests will be used to estimate overall significances where appropriate. Probability level of 5% (p<0.05) was considered significant for all statistical tests.

### Example 1

### SRY Controls Motor Function and Nigrostriatal Dopamine Levels in Male Rats

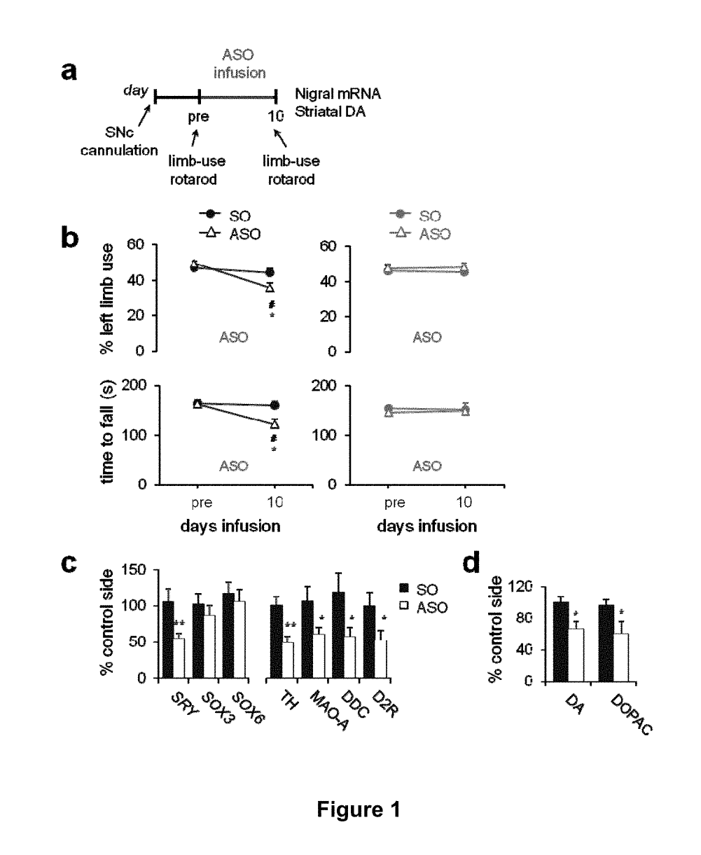

Repeated antisense oligonucleotide (ODN) treatment directed at the nigral SRY gene was provided to male and female rats with sense ODN negative controls. Motor function was assessed by the limb-use asymmetry and rotarod tests. Brains were then processed for nigral SRY, Sox-6, Sox-3, tyrosine hydroxylase (TH), DOPA decarboxylase (DDC), monamine oxidase-A (MAO-A) mRNA expression and striatal dopamine (DA) and dihydroxyphenylacetic acid (DOPAC) measurements. The results are shown in FIG. 1. Antisense treatment caused a significant reduction in motor function in male rats.

### Example 2

### Effect of SRY Antisense Treatment Prior to 6-OHDA Injection

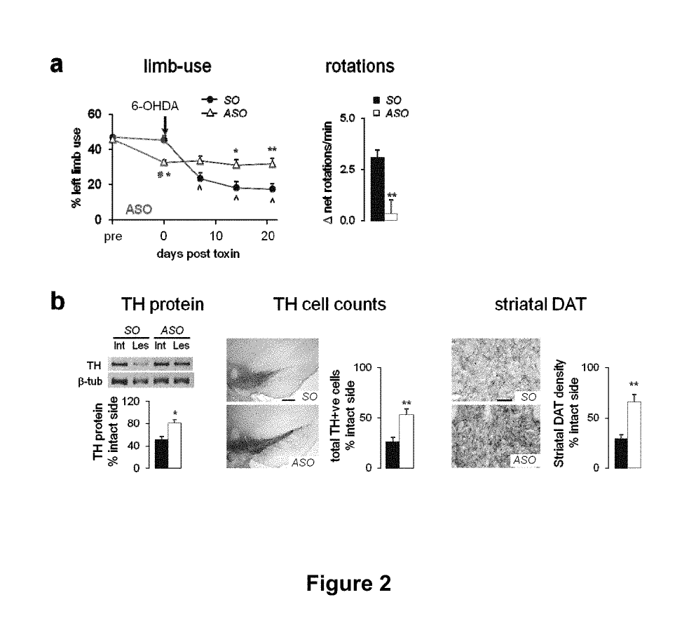

SRY antisense ODNs were repeatedly given to male rats along with negative sense ODN controls. Repeated SRY antisense treatment, before 6-OHDA injection, attenuated 6-OHDA-induced motor deficits and nigrostriatal degeneration in male rats. The results are shown in FIG. 2. The drug, 6-OHDA, induces Parkinsonian-like symptoms. SRY antisense treatment was shown to reduce development of these symptoms when given prior to 6-OHDA. Surviving neurons increased from 25% to 50%.

### Example 3

### Effect of SRY Antisense Treatment Following 6-OHDA Injection

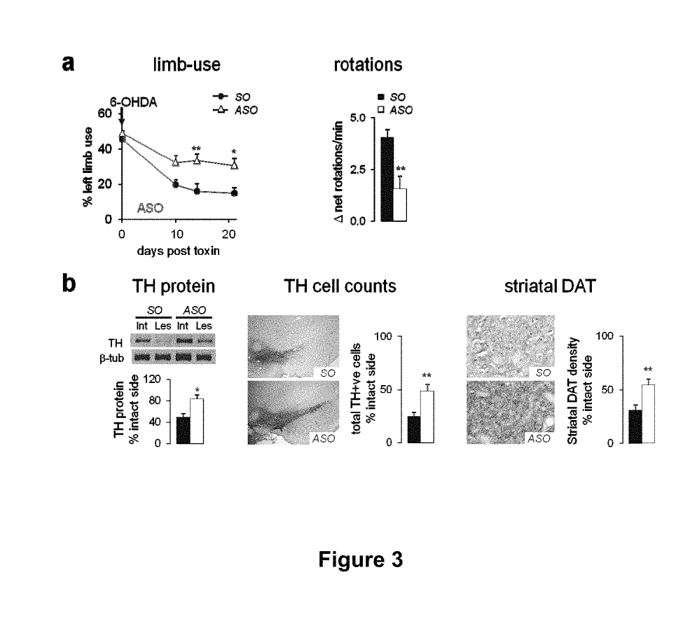

Example 2 was repeated except the antisense ODNs were administered after 6-OHDA treatment. Similar results were obtained. Repeated SRY antisense treatment, following 6-OHDA injection, attenuated 6-OHDA-induced motor deficits and nigrostriatal degeneration in male rats. The results are shown in FIG. 3.

### Example 4

### Effects of SRY Antisense Treatment Prior to Rotenone Injection

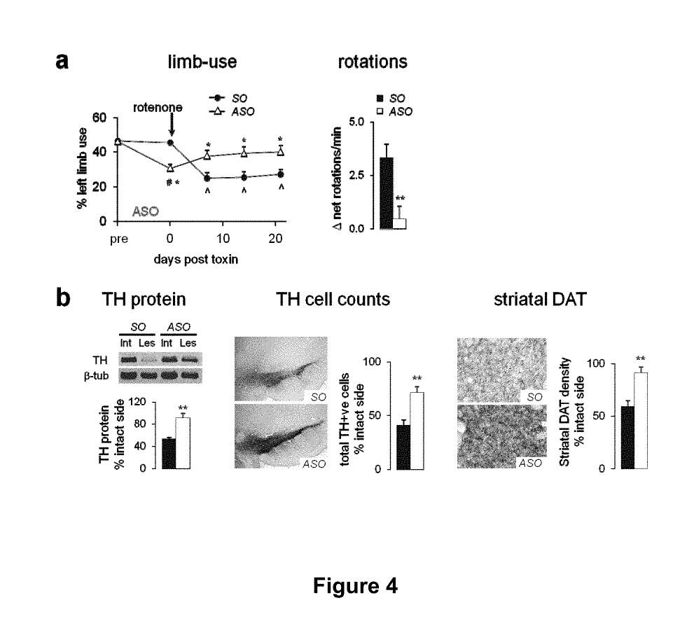

Example 2 was repeated using rotenone instead of 6-OHDA. Rotenone is another toxin which causes Parkinsonian-like symptoms. Repeated SRY antisense treatment, before rotenone injection, attenuated rotenone-induced motor deficits and nigrostriatal degeneration in male rats. The results are shown in FIG. 4.

### Example 5

### Effect of SRY Antisense Treatment on Female Rats

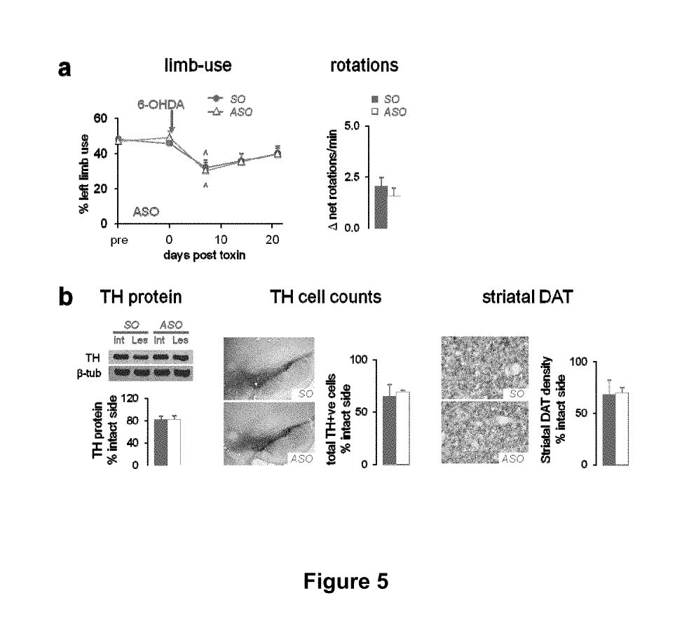

The effects of SRY antisense ODNs in female rats was tested. Repeated SRY antisense treatment, before 6-OHDA injection, did not affect 6-OHDA-induced motor deficits and nigral DA cell loss in female rats. The results are shown in FIG. 5. There was no substantial improvement which evidences the treatment is for male-biased disorders.

### Example 6

### Treatment Protocol

A treatment protocol is proposed for male subjects diagnosed with or having symptoms associated with a male-biased neurological disorder. Administration of an agent is proposed which down-regulates expression of the gene encoding SRY or which inhibits SRY function or activity include antisense oligonucleotides which target and down-regulate expression of SRY mRNA. It is proposed that in healthy brains, SRY controls dopamine-production and motor function. In a damaged brain, SRY up-regulation is deleterious. With progressive dopamine-producing cell loss, SRY neurons are selectively lost. Administration of the SRY-selective inhibiting agent is continued for as long as necessary to minimize progressive decline in patient health. For human use, the oligonucleotides in Table 4 represent one set of possible oligonucleotides. Another set of oligonucleotides comprising 20mer Gapmer binding to human SRY mRNA is provided in Table 5. The configuration is 10 nucleotides flanked with 10 2′-methoxyethyl-ribonucleotides with the bases phosphorothioated.

TABLE 4
Base sequences and positions of human SRY
antisense and sense oligodeoxynucleotide

| | | Target | |
|---|---|---|---|
| | | Region | SEQ |
| | | of | ID |
| ODN | Sequence¹ | mRNA | NO: |
| Antisense | 5′AGCAGAAGCATATGATTGCAT3′ | +1 to | 897 |
| ODN 1 | | +21 | |
| Antisense | 5′TAACATAGCAGAAGCATATGATT3′ | +5 to | 898 |
| ODN 2 | | +27 | |
| Antisense | 5′ATGATTGCATTGTCAAAAAC3′ | −10 to | 899 |
| ODN 3 | | +10 | |
| Control | 5′ATGCAATCATATGCTTCTGCT3′ | +1 to | 900 |
| (sense) | | +21 | |
| ODN 1 | | | |
| Control | 5′AATCATATGCTTCTGCTATGTTA3′ | +5 to | 901 |
| (sense) | | +27 | |
| ODN 2 | | | |
| Control | 5′GTTTTTGACAATGCAATCAT3′ | −10 to | 902 |
| (sense) | | +10 | |
| ODN 3 | | | |

¹underlined bases are phosphorothioated

TABLE 5
20mer Gapmer antisense oligonucleotides (ASO)
binding human SRY mRNA

| | | | SEQ |
|---|---|---|---|
| | | | ID |
| ODN | Sequence | Target | NO: |
| Target Site A | CAATGCAATCATATGCTTCT | Translation | 887 |
| | | start site | |
| ASOA | GUUACGTTAGTATACGAAGA | — | 888 |
| Target Site B | GAAAACAGTAAAGGCAACGT | mRNA loop | 889 |
| ASOB | CUUUUGTCATTTCCGUUGCA | — | 890 |
| Target Site C | ACCCATGAACGCATTCATCG | mRNA loop | 891 |
| ASOC | UGGGUACTTGCGTAAGTAGC | — | 892 |
| Target Site D | AAGCCACACACTCAAGAATG | mRNA loop | 893 |
| ASOD | UUCGGTGTGTGAGTTCUUAC | — | 894 |
| Target Site E | TAAAGGCCTTATTCATTTCA | PolyA signal | 895 |
| ASOE | AUUUCCGGAATAAGTAAAGT | — | 896 |

Those skilled in the art will appreciate that the disclosure described herein is susceptible to variations and modifications other than those specifically described. It is to be understood that the disclosure contemplates all such variations and modifications. The disclosure also enables all of the steps, features, compositions and compounds referred to or indicated in this specification, individually or collectively, and any and all combinations of any two or more of the steps or features or compositions or compounds.

### BIBLIOGRAPHY

Bonner and Laskey (1974) Eur. J. Biochem. 46: 83
Castillo et al. (1998) Mol Cell Neurosci, 11(1-2):36-46
Crooke et al. (1996) Ann. Rev. Pharm. Tox. 36:107-129
Czech et al. (2014) Endocrinolgy 155(7):2602-2612
Dewing et al. (2006) Current Biology 16(4):415-420
Ferrari et al. (1992) Embo J, 11(12):4497-506
Hargrave et al. (1997) Dev Dyn, 210(2):79-86
Harley et al. (1992) Science, 255(5043):453-6
Hunziker. et al. (1995) Mod. Syn. Methods 7:331-417
King and Weiss (1993) Proc Natl Acad Sci USA, 90(24):11990-4
Koopman et al. (1990) Nature, 348(6300):450-2
Koopman et al. (1991) Nature 351(6322):117-21
Kringlebach et al. (2007) Nature Reviews Neuroscience 8:623-635
Laudet et al. (1993) Nucleic Acids Res, 21(10) 2493-501
Lee et al. (2008) Brain 131:1574-1587
Lee et al. (2008) Synapse 62:310-313
Lindvall et al. (1987) Ann. Neurol. 22:457 468
Madrazo et al. (1987) New Engl. J. Med. 316:831 834
Malbon (2004) Front Biosci, 9:1048-58
Maxwell et al. (2005) Dev Biol, 282(2):467-79
Nasrin et al. (1991) Nature, 354(6351):317-20
Olanow and Tatton (1999) Annu Rev Neurosci, 22:123-44
Pevny and Lovell-Badge (1997) Curr Opin Genet Dev, 7(3):338-44
Remington's Pharmaceutical Sciences, 18th Edition, A. Gennaro, ed., Mack Publishing Co., Easton, Pa., 1990
Rex et al. (1997) Mech Dev, 66(1-2):39-53
Saunders-Pullman (2003) Endocrine, 21(1): 81-7
Simola et al. (2007) Neurotox Res 11(3-4):151-167
Uhlmann et al. (1990) Chem. Rev. 90:543
Uwanogho et al. (1995) Mech Dev, 49(1-2):23-36
Wang et al. (2013) Cell 153(4):910-918
Wegner (1999) Nucleic Acids Res, 27(6):1409-20

1. A method for the treatment or prophylaxis of a male-biased neurological disorder in a male subject, said method comprising administering to said male subject, an agent or vehicle carrying the agent which enters the brain and inhibits expression of the gene encoding sex-determining region, Y chromosome (SRY) or inhibits SRY function or activity in a dopamine-producing nerve cell in an amount effective to ameliorate symptoms, prevent development of the symptoms or minimize further progression of the symptoms of the neurological disorder.

2. The method of claim 1 wherein the dopamine-producing nerve cell is a dopaminergic neuron.

3. The method of claim 1 wherein the dopaminergic neuron is located in the substantia nigra pars compacta (SNc) of the brain.

4. The method of claim 3 wherein down-regulation of expression of the SRY gene or function or activity of the SRY protein reduces or inhibits progressive dopamine-producing cell loss.

5. The method of claim 4 wherein the neurological disorder is associated with loss of dopamine-producing cells.

6. The method of any one of claims 1 to 5 wherein the neurological disorder is selected from the list comprising Parkinson's disease, autism, epilepsy, attention deficit hyperactivity disorder (ADHD), psychosis, drug addiction and pain.

7. The method of claim 6 wherein the psychosis is schizophrenia.

8. The method of any one of claims 1 to 7 wherein the agent is a genetic molecule, small chemical molecule, peptide or a vehicle comprising same.

9. The method of claim 8 wherein the genetic molecule is an oligonucleotide which targets mRNA or DNA encoding SRY or a regulatory region thereof thereby reducing or inhibiting translation into active protein or expression into translatable mRNA.

10. The method of claim 9 wherein the oligonucleotide is selected from the list comprising short single stranded or double stranded RNA or DNA, iRNA, siRNA, hairpin RNA or DNA constructs.

11. The method of claim 9 or 10 wherein the oligonucleotide is selected from SEQ ID NO:88, 890, 892, 894, 896, 897, 898 and 899.

12. The method of claim 8 wherein the agent is a CRISPR/Cas agent.

13. The method of claim 10 or 11 or 12 wherein the oligonucleotide is produced by an expression vector.

14. The method of any one of claims 8 to 13 wherein the vehicle is a virus.

15. The method of any one of claims 1 to 14 wherein the agent is administered directly to the brain.

16. The method of claim 1 wherein the subject is a human male.

17. The method of claim 16 wherein the agent is administered in conjunction with deep brain stimulation.

18. A therapeutic protocol for treating or preventing a male-biased neurological disorder in a male subject, said protocol comprising:
(i) identifying and selecting the male subject based on behavior, genetic predisposition, symptoms or age or exposure to toxins or toxicants;
(ii) administering to said male subject, an agent or vehicle carrying the agent which enters the brain and inhibits expression of the gene encoding sex-determining region, Y chromosome (SRY) or inhibits SRY function or activity in a dopamine-producing nerve cell in an amount effective to ameliorate symptoms, prevent development of the symptoms or minimize further progression of the symptoms of the neurological disorder;
(iii) monitoring the symptoms and behavior of the male subject;
(iv) provide further agent or other medicaments or behavioral modification as required to maintain the health of the male subject.

19. The therapeutic protocol of claim 18 wherein the dopamine-producing nerve cell is a dopamine neuron.

20. The therapeutic protocol of claim 18 wherein the dopamine neuron is located in the substantia nigra pars compacta (SNc) of the brain.

21. The therapeutic protocol of claim 20 wherein down-regulation of expression of the SRY gene or function or activity of the SRY protein reduces or inhibits progressive dopamine-producing cell loss.

22. The therapeutic protocol of claim 21 wherein the neurological disorder is associated with loss of dopamine-producing cells.

23. The therapeutic protocol of any one of claims 18 to 22 wherein the neurological disorder is selected from the list comprising Parkinson's disease, autism, epilepsy, attention deficit hyperactivity disorder (ADHD), psychosis, drug addiction and pain.

24. The therapeutic protocol of claim 23 wherein the psychosis is schizophrenia.

25. The therapeutic protocol of any one of claims 18 to 24 wherein the agent is a genetic molecule, small chemical molecule, peptide or a vehicle comprising same.

26. The therapeutic protocol of claim 25 wherein the genetic molecule is an oligonucleotide which targets mRNA or DNA encoding SRY thereby reducing or inhibiting translation into active protein or expression into translatable mRNA.

27. The therapeutic protocol of claim 26 wherein the oligonucleotide is selected from the list comprising short single stranded or double stranded RNA or DNA, iRNA, siRNA, hairpin RNA or DNA constructs.

28. The therapeutic protocol of claim 26 or claim 27 wherein the oligonucleotide is selected from SEQ ID NO: 888, 890, 892, 894, 896, 897, 898 and 899.

29. The therapeutic protocol of claim 25 wherein the agent is a CRISPR/Cas agent.

30. The therapeutic protocol of claim 27 or 28 or 29 wherein the oligonucleotide is produced by an expression vector.

31. The therapeutic protocol of any one of claims 25 to 30 wherein the vehicle is a virus.

32. The therapeutic protocol of any one of claims 18 to 31 wherein the agent is administered directly to the brain.

33. The therapeutic protocol of claim 18 wherein the subject is a human male.

34. The therapeutic protocol of claim 33 wherein the agent is given in conjunction with deep brain stimulation.

35. Use of an agent which antagonizes SRY activity or function or SRY gene expression in the manufacture of a medicament to treat or ameliorate the symptoms of a male-biased neurological disorder in a male subject.

36. A method for the treatment or prophylaxis of a male-based neurological disorder in a male subject, said method comprising administering to the male subject, a CRISPR/Cas agent which enters the brain and disrupts the SRY gene thereby reducing its ability to express a functional protein in a dopaminergic nerve cell.
